(19) United States
(12) Patent Application Publication
(10) Pub. No.: US 2019/0247312 A1
(43) Pub. Date: Aug. 15, 2019

(54) PHARMACEUTICAL PREPARATION FOR DELIVERY OF PEPTIDES AND PROTEINS

(71) Applicant: Tamarisk Technologies Group LLC, Moorpark, CA (US)

(72) Inventors: Claudia M. Cardona, Danville, VA (US); Mark Ennis Ketner, Fuquay Varina, NC (US); Dakshinamurthy Devanga Chinta, Cary, NC (US); Carl R. Sahi, Coventry, CT (US); John M. Polidoro, Tolland, CT (US); James-Thomas Marion Gladden, Raleigh, NC (US)

(21) Appl. No.: 16/312,692

(22) PCT Filed: Jun. 27, 2017
(86) PCT No.: PCT/US17/39535

§ 371 Date: Dec. 21, 2018

Related U.S. Application Data
(60) Provisional application No. 62/355,072, filed on Jun. 27, 2016.

(51) Int. Cl. A61K 9/16; A61K 9/19; A61K 38/28

(52) U.S. Cl. CPC A61K 9/1652; A61K 9/1617; A61K 9/107; A61K 38/28; A61K 9/19

(57) ABSTRACT

Provided herein is a composite microparticle for oral delivery of an active agent to a subject. The microparticles are general in the form of self-sustaining bodies comprising a crosslinked polymer matrix and a plurality of emulsion droplets distributed throughout. The active agent is encapsulated in the emulsion droplets. A plurality of delivery enhancing moieties are presented on the exterior surface of said self-sustaining body and/or on the emulsion droplets. The microparticle is resistant to enteric degradation and will localize in the gastrointestinal tract of the subject without crossing the intestinal mucosa into the intestinal bloodstream.

### CROSS-REFERENCE TO RELATED APPLICATIONS

The present application claims the priority benefit of U.S. Provisional Patent Application Ser. No. 62/355,072, filed Jun. 27, 2016, entitled PHARMACEUTICAL PREPARATION FOR DELIVERY OF PEPTIDES AND PROTEINS, and incorporated by reference in its entirety herein.

### BACKGROUND OF THE INVENTION

### Field of the Invention

The present invention relates generally to compositions of, and methods for the manufacturing and use of, effective drug delivery formulations.

### Description of Related Art

Many pharmaceutical and nutraceutical products have heretofore been unamenable to oral ingestion. Barriers to oral administration of these therapeutic agents include, but are not limited to, a need to carefully regulate or target dosage, an inability to readily absorb these agents from the gastrointestinal (GI) tract, the denaturation and digestion of protein drugs by gastric juices, or the requirement for hydrophobic/hydrophilic stabilizers in some compositions.

However, oral ingestion is generally viewed by most clinicians as the ideal method of delivery for lowering the equally important barrier of patient compliance. Therefore, there exists an unmet need for delivery systems capable of overcoming these and other similar challenges.

### SUMMARY OF THE INVENTION

The invention is broadly concerned with polymer matrix-emulsion combination particles for the purpose of improving targeting and stability of active agents within the particle as well as, very particularly in the oral delivery of active agents to a subject. More specifically, delivery-enhanced particles comprising solid-in-oil-in-water emulsion droplets for the purpose of orally delivering an active pharmaceutical ingredient are described. It further relates to the addition of delivery-enhancing moieties to the matrix-emulsion combination to effect higher absorption of active pharmaceutical ingredients.

Broadly, microparticles comprising a self-sustaining body comprising an active agent encapsulated within emulsion droplets are described herein. The emulsion droplets further comprise a plurality of optional delivery enhancing moieties, wherein at least a portion of said delivery enhancing moieties are presented on the exterior surfaces of said emulsion droplets.

In one aspect, composite microparticles for oral delivery of an active agent to a subject are provided. The composite microparticles generally comprise a self-sustaining body comprising a crosslinked polymer matrix and a plurality of emulsion droplets distributed throughout the polymer matrix. The active agent is encapsulated in the emulsion droplets. The composite microparticles further comprise a plurality of delivery enhancing moieties, wherein at least a portion of the delivery enhancing moieties are presented on the exterior surface of the self-sustaining body and/or on the emulsion droplets. Advantageously, the microparticle is resistant to enteric degradation and will localize in the gastrointestinal tract of the subject without crossing the intestinal mucosa into the intestinal bloodstream.

The ability to administer insulin to diabetic patients orally remains an obstacle due to the need to pass a protein hormone through the inhospitable environment of the stomach and duodenum where acids and proteases abound that would readily inactivate and digest the drug prior to its arrival into the blood stream. However, making this drug available to the public in such a form has the potential to transform the nature of treatment by improving patient compliance and even reducing the reliance on glucose testing devices as insulin could be taken orally with each meal according to its size and composition. This would also lower the barriers that young diabetics have in learning to overcome needle-aversion and self-administer their medication more easily.

Compounding the problem of digestive inactivation is that while the pancreas secretes many enzymes into the duodenum directly, insulin, like other pancreatic hormones is not secreted into the gastrointestinal (GI) system, but is directly taken up by the blood via a highly fenestrated capillary network surrounding beta-islet cells. Therefore, the intestinal epithelial cells are not natively equipped to take up insulin even if it could be delivered there in an effective form.

A second aspect of the invention is then facilitating the uptake of APIs from the GI tract in the absence of native receptors. To accomplish this, the polymer matrix particles are further equipped with surrogate ligands based on the presence of cognate receptors on GI epithelial cells.

To this end, formulating insulin for oral delivery provides not only proof of principle for protecting a protein drug within the GI environment, but also demonstrating the efficacy of using specific GI receptor ligands to mediate uptake of particles into systemic circulation.

An emulsion consists of two immiscible liquids in two phases, a globular dispersed phase homogeneously distributed in a continuous phase. Because emulsions are thermodynamically unstable, surfactants are used to stabilize the system, and also assist with delivery of the active agents. For this application, a multiple emulsion technique was employed. These are emulsions of emulsions and consist of oil-in-water or water-in-oil emulsions dispersed in an additional liquid media. This results in either an oil-in-water-in-oil (O/W/O) or a water-in-oil-in-water (W/O/W) emulsion. A third type of multiple emulsion in the present disclosure is a solid-in-oil-in-water (S/O/W) emulsion.

Microemulsions/nanoemulsions (those with average droplet size usually between 50 to 1000 nm) can be used to increase the solubility and bioavailability in addition to controlling the release of the active agent in the body. These emulsions are increasingly being used as an alternative to liposome delivery mechanisms. In addition to simple delivery of insoluble drugs, these emulsions can now be targeted for delivery to various tissues and receptors despite the absence of native receptor molecules for the target itself. These targeted approaches further improve outcomes by leading to increased efficacy, the potential for lower dosing, and reduction of side effects.

The encapsulation formulations contemplated herein are suitable for the encapsulation and subsequent delivery to the GI tract of a broad spectrum of hydrophobic and hydrophilic biologically active, therapeutic or nutritionally-useful molecules such as, but not limited to, those described elsewhere herein.

Thus, provided herein are also compositions comprising a therapeutically-effective amount of a plurality of microparticles according to embodiments of the invention dispersed in a pharmaceutically acceptable carrier.

Methods of orally administering an active agent to a subject in need thereof are also described. The methods generally comprise administering a therapeutically effective amount of microparticles according to embodiments of the invention to the subject. Use of a composition according to administer a therapeutically effective amount of active agent to a subject in need thereof is also described.

Also provided herein are methods of forming a composite microparticle. The methods generally comprise combining a polymer suspension with an active agent emulsion to yield a mixture. The polymer suspension comprises a polymer matrix precursor dispersed in a solvent system. The active agent emulsion comprises an active agent encapsulated in an emulsion. Either or both of the polymer suspension and/or the active agent emulsion further comprises a plurality of delivery enhancing moieties. The polymer matrix precursor in the mixture is then crosslinked to yield a crosslinked polymer matrix in the form of a self-sustaining body having an exterior surface and a plurality of emulsion droplets comprising the active agent distributed throughout. In the resulting microparticle, at least a portion of the delivery enhancing moieties are presented on the exterior surface of the self-sustaining body and/or on the emulsion droplets. Advantageously, the self-sustaining body is resistant to enteric degradation but capable of localization in the gastrointestinal tract of a subject without crossing the intestinal mucosa into the intestinal bloodstream.

### BRIEF DESCRIPTION OF THE DRAWINGS

FIG. 1 provides structural representations of exemplary carotenoids.

FIG. 2 is a depiction of one embodiment of a carotenoid-bound alginate polymer.

FIG. 3 is an illustration of the apparatus used to generate HCl gas.

FIG. 4 is an illustration of the distillation apparatus used to remove acetone from the Beta-chloro-carotene (BCC.)

FIG. 5A is an illustration of a delivery-enhanced particle comprising active agent emulsion droplets.

FIG. 5B is a scanning electron micrograph (SEM) of a delivery-enhanced alginate particle comprising active agent emulsion droplets.

FIGS. 6A and 6B are SEMs of the inside of the delivery-enhanced alginate particle comprising active agent emulsion droplets.

FIG. 7A is a plot of blood insulin concentration following administration of 5.0 IU/kg subcutaneous (s.c.) insulin.

FIG. 7B is a plot of blood glucose concentration following administration of 5.0 IU/kg s.c. insulin.

FIG. 8 is a plot of blood insulin and glucose concentrations following administration of zinc-complexed insulin in the emulsion particles with BCC (TA-3) at 50 IU/kg p.o.

FIG. 9A is a plot of blood insulin and glucose concentrations following administration of zinc-complexed insulin in the emulsion particles without BCC (TA-4) at 50 IU/kg p.o.

FIG. 10A is a plot of blood insulin concentrations following administration of uncomplexed insulin in the emulsion particles with BCC (TA7) at 50 IU/kg p.o. for three exemplary animals.

FIG. 10B is a plot of blood glucose concentrations following administration of uncomplexed insulin in the emulsion particles with BCC (TA7) at 50 IU/kg p.o. for three exemplary animals.

FIG. 11A is a plot of blood insulin concentrations following administration of control s.c. injections of 5 IU/kg unformulated insulin.

FIG. 11B is a plot of blood glucose concentrations following administration of control s.c. injections of 5 IU/kg unformulated insulin.

FIG. 12 is a plot of blood insulin and glucose concentrations following administration of microemulsion particles with zinc-complexed insulin (TA-1) at 33 IU/kg p.o.

FIG. 13 is a plot of blood insulin and glucose concentrations following administration of zinc-complexed insulin dispersed in alginate matrix (no emulsion; TA-5) at 33 IU/kg p.o.

FIG. 14A is a plot of blood insulin and glucose concentrations following administration of nanoemulsion particles with zinc-complexed insulin (TA-6) at 33 IU/kg p.o. in an exemplary animal in this group.

FIG. 14B is a plot of blood insulin and glucose concentrations following administration of nanoemulsion particles with zinc-complexed insulin (TA-6) at 33 IU/kg p.o. in an exemplary animal in this group.

FIG. 15A is a plot of blood insulin and glucose concentrations following administration of microemulsion particles with zinc-complexed insulin, and high fat diet (TA-7) at 33 IU/kg p.o. in an exemplary animal in this group.

FIG. 15B is a plot of blood insulin and glucose concentrations following administration of microemulsion particles with zinc-complexed insulin, and high fat diet (TA-7) at 33 IU/kg p.o. in an exemplary animal in this group.

FIG. 15C is a plot of blood insulin and glucose concentrations following administration of microemulsion particles with zinc-complexed insulin, and high fat diet (TA-7) at 33 IU/kg p.o. in an exemplary animal in this group.

FIG. 16A is a plot of blood insulin concentrations following administration of zinc-complexed insulin in emulsion particles in several animals in the study in comparison to administration of insulin s.c.

FIG. 16B is a plot of blood glucose concentrations following administration of zinc-complexed insulin in emulsion particles in several animals in the study in comparison to administration of insulin s.c.

FIG. 17 is a plot of blood insulin concentrations from Examples 3 (where the delivery particles were designed for a multi-hour extended release and absorption profile, dotted line) and 5 (where the delivery particle parameters were adjusted for a greater burst release profile to better track the subcutaneous control profile, dark line) in comparison with an s.c. control (light line).

### DETAILED DESCRIPTION

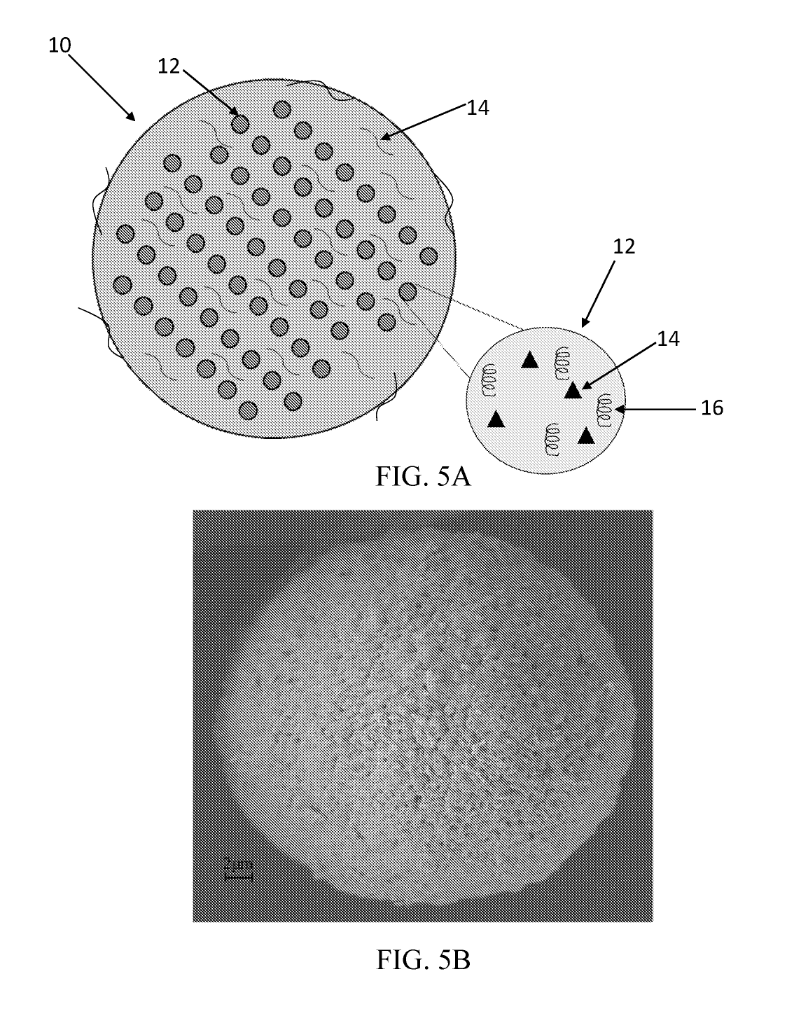

The presently claimed and disclosed inventive concept(s) relates generally to pharmaceutical and nutraceutical products, more particularly to improved encapsulated pharmaceutical and nutraceutical active agents, methods of their production, and methods of their use. FIG. 5A is a representative schematic of an enhanced-delivery particle 10 comprising emulsion droplets 12 and optional delivery-enhancing moieties 14 encapsulating an active agent 16. Delivery-enhancing moieties 14 are depicted in the particle matrix as well as in the emulsion droplets 12. It will be appreciated that delivery-enhancing moieties may optionally be present only in the particle matrix or in the emulsion droplets (but not both). In some cases, delivery-enhancing moieties will be present in both. In some cases, the same type of delivery-enhancing moiety will be used in both the particle matrix and in the emulsion droplets. In some cases, a different type of delivery-enhancing moiety will be used in each of the particle matrix and in the emulsion droplets. It is also appreciated that combinations of more than one type of delivery-enhancing moiety may be used in the various embodiments. The resulting particle sizes range from about 1 μm to about 100 μm.

In one or more embodiments, the encapsulation formulations are self-sustaining bodies, each comprising a cured or crosslinked polymer matrix and a delivery-enhancing moiety. Particle sizes (e.g., diameter) can range from about 100 nm and up, preferably about 500 nm up to about 5 mm. In some embodiments, larger particles are desired with average diameters (i.e., maximum surface-to-surface dimension) for particles, as formed, ranging from about 1 to about 5 mm. In some embodiments, smaller particles are desired with average diameters for particles, as formed, ranging from about 1 μm to about 1000 μm, more preferably from 1 μm to about 200 μm, and even more preferably from about 1 μm to about 20 μm. Exemplary delivery enhancing moieties are selected from the group consisting of (1) medium or long chain fatty acids, (2) isoprenoids, (3) vitamins, (4) signal peptides, and combinations thereof. One or more active agents can be encapsulated (i.e., completely contained and/or enveloped) in emulsion droplets, which are distributed throughout the polymer matrix. More specifically, the active agents are encapsulated in solid-in-oil-in-water multiple emulsion droplets, which are distributed throughout the polymer matrix. The solid-in-oil-in-water emulsion droplets comprise the active agent, an oil, at least one surfactant, and water. One or more delivery-enhancing moieties can also be included in the solid-in-oil-in-water emulsion droplets. Droplet sizes (e.g., diameters) can range from about 50 nm to about 1000 nm. In some embodiments, the invention includes nanoemulsions with droplet sizes ranging from about 300 to about 900 nm. In some embodiments, the invention includes microemulsions with droplet sizes ranging from about 100 to about 300 μm.

In general, the encapsulation formulation is formed by preparing the emulsion droplets, which are then mixed with a polymer matrix precursor and delivery-enhancing moieties, followed by curing/crosslinking the polymer matrix precursor under conditions yielding discrete, self-sustaining enhanced-delivery particles, with the delivery-enhancing moiety and solid-in-oil-in-water emulsion droplets substantially uniformly distributed throughout the crosslinked polymer matrix. In some embodiments, one or more delivery-enhancing moieties may also be on (and in some cases extend from) the exterior surface of the crosslinked polymer matrix/particle. The resulting particle(s) can be used as part of an oral dosage form for administering the active agent to a subject.

The solid-in-oil-in-water emulsion droplets comprise the active agent, an oil, at least one surfactant, and water (purified, distilled, DAW, etc.). Suitable oils for use in the invention include those that are pharmaceutically-acceptable, especially for oral use, including fatty acids, triglycerides, and the like, with plant-based oils being particularly preferred (e.g., vegetable or seed-based oils); however, animal and/or mineral oils may also be used. Exemplary oils include canola, safflower, olive, sesame, almond, sunflower, corn, soybean, rapeseed, hempseed, grapeseed, coconut, palm, pumpkin seed, avocado, castor, or peanut oil, and mixtures thereof. In some embodiments, the oil is one or more oils selected from the group consisting of pumpkin seed, sesame, almond, peanut, sunflower, rapeseed, and/or olive oils. As used herein, the term “pharmaceutically-acceptable” means not biologically or otherwise undesirable, in that it can be administered to a subject, cells, or tissue, without excessive toxicity, irritation, or allergic response, and does not cause undesirable biological effects or interact in a deleterious manner with the other constituents of the composition in which it is contained. Pharmaceutically-acceptable ingredients include those acceptable for veterinary use as well as human pharmaceutical use. It will also be appreciated that in some contexts, such as in the delivery of chemotherapeutic agents to cancer cells, toxic ingredients may be considered “pharmaceutically-acceptable.”

Suitable surfactants for use in the invention include nonionic surfactants, such as sorbitan esters (such as sorbitan monooleate, e.g., Polysorbate 80, Span 80), polyethoxylated sorbitan esters (such as ethoxylated sorbitan monolaurate, e.g., Polysorbate 20, Tween 20), and the like.

As mentioned, one or more delivery-enhancing moieties can also be included in the solid-in-oil-in-water emulsion droplets. Exemplary delivery-enhancing moieties are described in more detail below.

The solid-in-oil-in-water emulsion droplets are prepared by combining the active agent with the oil, surfactant(s), and water. In some embodiments, the active agent may need to be solubilized first to facilitate its dispersion in the emulsion. For example, in the case of certain proteins, such as insulin, the active agent may need to be dissolved in an acid (e.g., HCl), followed by adjusting the pH between 4.5-7.0 using additional acid (e.g., HCl) or base (e.g., NaOH), as needed. The active agent is then precipitated out of the solution, such as by using centrifugation, before being used to create the emulsion droplets. In general, a primary emulsion is prepared by mixing a nonionic surfactant (e.g., Span 80) with the selected oil. If desired, a delivery-enhancing moiety is added to the mixture. The components are homogenized/mixed thoroughly to create the primary emulsion. The prepared active agent is then mixed with the primary emulsion, followed by thorough mixing to uniformly disperse the active agent in the emulsion. In one or more embodiments, mixing is carried out in an ice bath. The resulting secondary emulsion (with the active agent) is then stabilized using an aqueous solution containing a second nonionic surfactant (e.g., Tween 20). In one or more embodiments, mixing is carried out in an ice bath to homogenize the mixture and stabilize the emulsion droplets. The foregoing process results in the solid-in-oil-in-water multiple emulsion.

The solid-in-oil-in-water multiple emulsion is then mixed with a polymer matrix precursor, in which the emulsion coalesces into distinct, individual droplets dispersed throughout the polymer precursor. Each droplet comprises the active agent encapsulated in the emulsion droplet with the oil, nonionic surfactant(s), and optional delivery-enhancing moiety on or at the surface of the droplet (as well as distributed therein).

Various swellable and/or biodegradable polymers can be used for the polymer matrix. Exemplary polymer systems include, without limitation, polyvinyl alcohol, poly(ethylene glycol), polylactic acid, poly-L-lactic acid, polycaprolactone, polyglycolic acid, poly(lactic-co-glycolic acid), polyhydroxyalkanoate, poly [N-(2-hydroxypropyl) methacrylamide], hyaluronic acid, gelatin, cellulose derivatives (e.g., carboxymethyl cellulose, hydroxypropyl methylcellulose), carbomers, and polysaccharides such as xanthan gum, chitosan, alginate, and/or pectin. Combinations of the foregoing polymers can also be used. Preferably, the polymer matrix precursor has gel-forming capabilities for creation of discrete gel particles prior to crosslinking/curing of the matrix into solid particles. Thus, the solid-in-oil-in-water multiple emulsion described above is mixed with the polymer precursor gel prior to particle formation.

In one or more embodiments, the polymer matrix comprises (consists essentially or even consists of) swellable and/or biodegradable polymers such as sodium, potassium, or calcium alginate (and optionally contains another polymer such as, but not limited to, carrageenan, xanthan gum, agar-agar, and/or chitosan). In preferred embodiments, calcium alginate (obtained for example by reaction of sodium alginate as the polymer precursor with a calcium salt such as CaCl₂)) is the primary constituent of the crosslinked polymer matrix. In the configuration contemplated herein, it forms multiple cross-linked helix-helix aggregates resulting in superior encapsulation strength. In alternative embodiments, a minor portion of the matrix may further comprise quantities of one or more other natural gums for cross-linking and thickening, such as, but not limited to, carrageenan, agar agar, guar gum, xanthan gum, and/or chitosan.

In one or more embodiments, the polymer matrix is initially made using sodium alginate (or potassium alginate) as the polymer base building block which is then, in one embodiment, converted to a more stable calcium form through ionic exchange. For example, upon contacting sodium or potassium alginate aggregates with an aqueous solution of calcium chloride (e.g., 1% to 40% by weight, preferably 1-24% or any effective concentration) or calcium acetate, for example, the sodium (or potassium) of the alginate aggregates is replaced by calcium. This reaction occurs rapidly at room temperature (e.g., 20-25° C.) or below resulting in the formation of helix-helix loaded aggregates which rapidly separate from the aqueous medium in the form of a coacervated precipitate. The resulting particle is then dried to a moisture content of <10%, and preferably <6%, forming an encapsulated product which is resistant to gastric fluid (i.e., enterically resistant) but allows particles to swell and release active agents at conditions consistent with the environment of the targeted region of the GI tract.

In addition to converting loaded aggregates to a water-insoluble form, calcium plays another key role in the molecular configuration of the oral dosage form. In particular, it causes cross-linkage of neighboring polymer molecules through calcium cross-linking. The resulting stability of the delivery system is set in a three-dimensional substantially-spherical configuration which serves not only to hold active agents more securely, but in the protection of the active agent from oxidative degradation, UV degradation, moisture degradation in addition to a vast number of other environmental stresses.

During curing, the exchange of sodium (or potassium) by calcium particularly enhances multiple cross-linkage formation between subunit molecules of the polymer enabling precipitation. In embodiments utilizing alginate as the polymer, the alginate portion may range from about 5%-99.5% by weight in the final particle while the other gums or copolymers (e.g., carrageenan, agar-agar, guar gum, xanthan gum, and/or chitosan) may optionally make up to about 20%, and preferably from about 0.1-5% of the final particle. In other embodiments, the sodium (or potassium) alginate is left in a gel form rather than in a precipitated form, which can then be contained in a suitable dosage form (e.g., gel cap). The sodium, potassium, or calcium alginate polymer used, as above, may have a molecular weight ranging from 10,000-600,000 Daltons, or preferably 100,000-400,000 Daltons, or more preferably 300,000-320,000 Daltons, and still more preferably 305,000 Daltons.

Sodium alginate (and potassium alginate) has the unusual ability to form a gel upon agitation within cold water which will not solidify upon standing. The gels thus formed have a high encapsulation affinity, meaning the ability of the alginate molecule to surround and wind itself around another molecule. In this way a three-dimensional network builds up in which double helices form junction points of the polymer chains thus allowing for the formation of multiple helix-helix aggregates which wind around and encapsulate the emulsion droplets. Sodium alginate is typically obtained by extraction from brown algae and is widely used within the food industry to increase product viscosity and as an emulsifying agent. Alginates are linear unbranched polymers containing (1→4)-linked D-mannuronic acid (M) and its epimer α-(1→4)-linked L-guluronic acid (G). D-mannuronic acid residues are enzymatically converted to L-guluronic after polymerization.

Alginates are not random copolymers but, according to the algal source, comprise blocks of similar alternating residues, each of which have different conformational preferences and behavior. The alginate polymer may comprise, for example, homopolymeric blocks of consecutive G-residues, or consecutive M-residues, or alternating M- and G-residues or randomly organized blocks of G- and M-residues. For example, the M/G ratio of alginate from Macrocystis pyrifera is about 1.6, whereas that from Laminaria hyperborea is about 0.45.

Although sodium alginate (or potassium alginate) in itself is very effective in molecular encapsulation activity, an even higher encapsulation affinity to the active agent therein can be obtained through the addition of 0.1 to 1% to 2% to 3% to 4% to 5% or more of a thickening agent or cross-linking agent such as carrageenan, xanthan gum, and/or agar-agar to the alginate. Agar-agar is extracted from the cell membranes of some species of red algae, particularly those from the genera Gelidium and Gracilaria. Historically agar-agar has chiefly been used as an ingredient in desserts, especially in Japan agar-agar comprises a mixture of agarose and agaropectin. Agarose is a linear polymer, of molecular weight about 120,000, based on the -(1→3)-β-D-galactopyranose-(1→4)-3, 6-anhydro-α-L-galactopyranose unit. Agaropectin is a heterogeneous mixture of smaller molecules that occur in lesser amounts. Their structures are similar but slightly branched and sulfated, and they may have methyl and pyruvic acid ketal substituents.

Delivery-enhancing moieties are also mixed with the polymer precursor, preferably, after the addition of the solid-in-oil-in-water multiple emulsion. Embodiments of the invention include the incorporation of selected delivery enhancing moieties which interact with the polymers directly in ionic, covalent, or supramolecular manners or indirectly via physical entrapment within the polymer matrix and/or within the emulsion droplets. Delivery-enhancing moieties used herein include molecules capable of facilitating the absorption and uptake of active agents by cells located throughout the GI tract and the subsequent systemic bioavailability of these agents. The delivery enhancing moieties can be first dispersed in a suitable aqueous- or oil-based carrier to facilitate even distribution of the delivery enhancing moieties upon combining with the polymer or emulsion.

Exemplary delivery-enhancing moieties for use in the various embodiments of the invention include long and medium chain fatty acids which generally have a chain length varying from 6-28 carbon atoms. For use herein, long chain fatty acids, especially fusogenic lipids (unsaturated fatty acids and monoglycerides such as oleic acid, linolenic acid, linoleic acid, monoolein, phosphatidylserine, and phosphatidylethanolamine) provide useful carriers to enhance targeted delivery and uptake of the active agents contemplated herein. Medium chain fatty acids (C6 to C12) may also be used to enhance targeted delivery and uptake of the particle or payload encapsulated thereby. Other medium and long chain fatty acids that can be used as delivery enhancers herein include, but are not limited to myristoleic acid, palmitoleic acid, oleic acid, alpha-linolenic acid, arachidonic acid, eicosapentaenoic acid, erucic acid, and docosahexaenoic acid. Examples of naturally-occurring fatty acids which may be used in the presently claimed and disclosed inventive concept(s) include but are not limited to C8:0 (caprylic acid), C10:0 (capric acid), C12:0 (lauric acid), C14:0 (myristic acid), C16:0 (palmitic acid), C16:1 (palmitoleic acid), C16:2, C18:0 (stearic acid), C18:1 (oleic acid), C18:1-7 (vaccenic), C18:2-6 (linoleic acid), C18:3-3 (alpha-linolenic acid), C18:3-5 (eleostearic), C18:3-6 (delta-linolenic acid), C18:4-3, C20:1 (gondoic acid), C20:2-6, C20:3-6 (dihomo-gamma-linolenic acid), C20:4-3, C20:4-6 (arachidonic acid), C20:5-3 (eicosapentaenoic acid), C22:1 (docosenoic acid), C22:4-6 (docosatetraenoic acid), C22:5-6 (docosapentaenoic acid), C22:5-3 (docosapentaenoic), C22:6-3 (docosahexaenoic acid) and C24:1-9 (nervonic). Highly preferred unbranched, naturally occurring fatty acids are those with from 14 to 22 carbon atoms. In addition, sodium salts of medium and long chain fatty acids are effective delivery enhancing molecules.

Examples of isoprenoid-type delivery enhancing moieties used herein include, but are not limited to, lycopene, limonene, gamma-tocotrienol, geraniol, carvone, farnesol, geranylgeraniol, squalene, and other linear terpenoids, carotenoids, taxol, vitamin E, vitamin A, beta-carotene, Coenzyme Q10 (ubiquinone), astaxanthin, zeaxanthin, lutein, citranxanthin, beta-chloro-carotene, canthaxanthin, and oxygenated forms thereof. Lycopene is an isoprenoid pigment responsible for the bright red color of tomatoes and other red fruits and vegetables. As a carotene, lycopene is an important intermediate in the biosynthesis of many carotenoids, such as a beta carotene. Lycopene is a symmetrical tetraterpene assembled from 8 isoprene units and absorbed in the intestine via, at least, the scavenger receptor class B type I. Beta-carotene is another isoprenoid compound used herein as a delivery enhancing molecule. Like lycopene, it is also absorbed by, at least, the scavenger receptor class B type I.

Delivery enhancing moieties contemplated herein also include vitamins, as well as signal peptides.

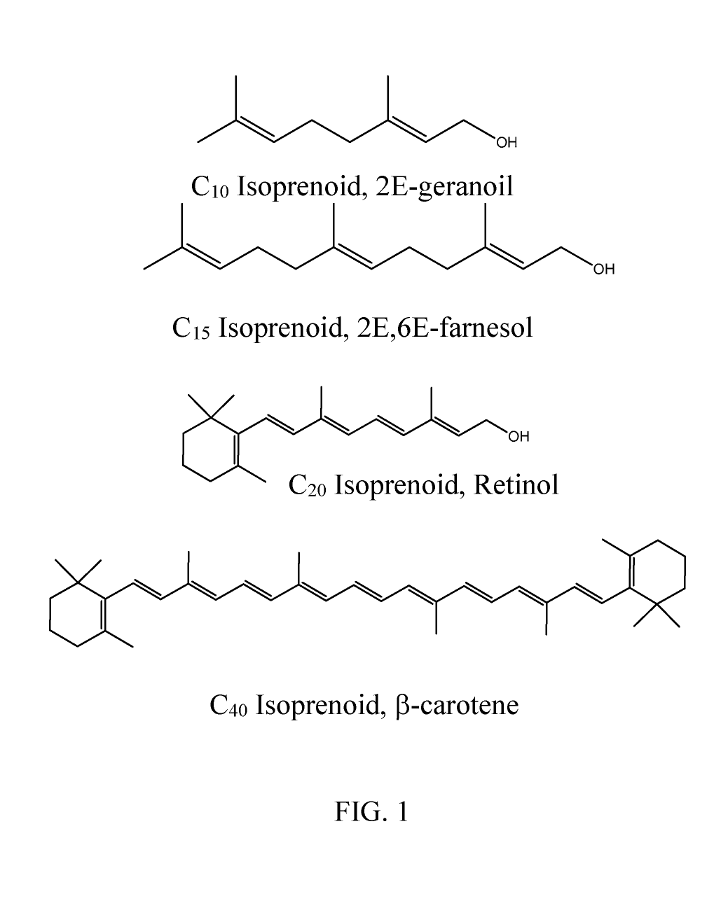

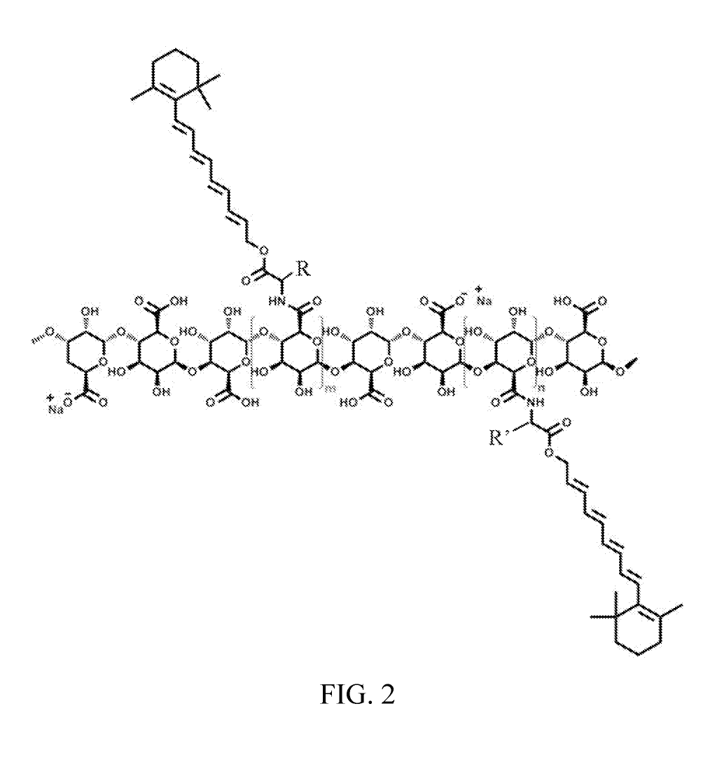

The incorporation of the delivery enhancing moieties into the chosen swellable and/or biodegradable polymers can be achieved through non-covalent, covalent, or supramolecular bonds as appropriate according to the functional groups of the polymer and the delivery enhancing moieties. In one or more embodiments, carotenoid moieties, containing one or more hydroxyl (—OH) group(s), are esterified with an amino acid, and then the amino group of the amino acid (—NHR) is coupled to the carboxylic acid units of the alginate (—COOH). In various embodiments of the invention, the carotenoid can encompass any of available hydroxylated-carotenes such as, but not limited to, Retinol, Lutein, Zeaxanthin, Cryptoxanthin, Hydroxyechinenone, Astaxanthine, Diatoxanthin, Dinoxanthin, Antheraxantin, Diadinoxanthin, Echinenone, Neoxanthin, Flavoxanthin, Violaxanthine, Rubixanthin, Fucoxanthin, and isomers thereof. See Figure (FIG. 1 for an illustration of example compounds. FIG. 2 illustrates by way of non-limiting example, alginate polymer modified by the covalent attachment of retinol.

Alternatively, the delivery enhancing moieties can be associated with the polymer through non-covalent attachments (e.g., ionic bonds, van der Waals interactions, and the like). The delivery enhancing moieties may also simply be physically restrained by the polymer matrix due to entrapment therein. It will be appreciated that a combination of approaches can be used to associate the delivery enhancing moieties with the polymer, which may depend on the particular polymer as well as the delivery enhancing moiety utilized in the particles.

Excipients regulating time-release (referred to herein a “time-release excipients”) can optionally be used in the polymer matrix. They interact with the polymers and/or the active agent in a manner causes the components of the particle to dissociate/diffuse differently (faster or slower) over time. Examples of time-release excipients which can be used in the presently claimed and disclosed inventive concept(s) includes, but are not limited to, polar molecules such as polyhydroxyl molecules, which include monosaccharides (glucose, fructose, galactose, xylose, mannose, tagatose), disaccharides (trehalose, lactose, sucrose, maltose, isomaltose, trehalulose), sugar alcohols (erythritol, glycerol, isomalt, lactitol, maltitol, mannitol, sorbitol, xylitol), polyoxy molecules (polyethylene glycols (PEGs), Sorbitan, Tween, Polysorbate, poloxamers), and/or chitosan.

Regardless, the enhanced-delivery particle emulsion droplets are able to encapsulate many classes or types of active agents including, but not limited to, therapeutic or nutraceutical agents such as antibiotics, antivirals, oncological agents, anti-lipids, antihypertensives, cardiac drugs, antidiabetic agents, vitamins, minerals, proteins, peptidomimics, monoclonal antibodies, and/or RNA or DNA molecules. More specifically the encapsulated active agent of the presently claimed and disclosed inventive concept(s) may be selected, for example, from anabolic agents (e.g., boldandiol, ethylestrenol, mibolerone, nandrolone, oxymetholone, stanozol, and testosterone); antibacterial/antibiotics (e.g., aminoglycosides including: amikacin, apramycin, dihydrostreptomycin, gentamicin, kanamycin, neomycin, spectinomycin, vancomycin; cephalosporins including: cefaclor, ceftazidime, cephalexin, cephalothin; clindamycin; chlorhexidine, fatty acid monoesters, such as glycerol monolaurate; fluoroquinolones including enroflaxacin, ciprofloxacin; macrolides including erythromycin, lincomycin, tylosin; penicillins including amoxicillin with and without potentiators, ampicillin, hetacillin, ticarcillin; tetracycline and analogues; sulfonomides with or without potentiators including sulfachloropyridazine, sulfadimethoxine, sulfamethazine, and sulfaquinoxaline); antifungals (e.g., miconazole, itraconazole, griseofulvin, glycerol mono-laureate, and metronidazole); anti-cancer agents (e.g., actinomycin-D, cisplatin, cytarabine, doxorubicin, 5-fluorouracil, methotrexate, pergolide, purine analogues, oncovin, vinblastine, and vincristine); antidotes and reversing agents (e.g., atropine, 2-PAM, naloxone, and nalorphine HCI, yohimbine, (atipamezole); antihistamines (e.g., cromolyn sodium, diphenhydramine, pyrilamine, and tripelennamine); antipyretics (e.g. acetaminophen); non-steroidal anti-inflammatory drugs (NSAIDs), (e.g., flunixin meglumine, acetylsalicylic acid, ibuprofen, ketoprofen, meclofenamic acid, naproxen, phenylbutazone, and zileuton); steroidal anti-inflammatory drugs (e.g., beclomethasone, budesonide, dexamethasone, flumethasone, flunisolide, fluticasone, isoflupredone, prednisolone, and triamcinolone); anti-thrombotics (e.g., acetylsalicylic acid); anti-tussives (e.g., narcotic analgesics, dextromethorphan, and pholcodine); bronchodilators (e.g., atropine, albuterol, clenbuterol, pirbuterol, salmeterol, fenoterol, aminophylline, glycopyrrolate, terbutaline, and theophylline); parasympathomimetics (e.g., bethanechol); anticholinergics (e.g., atropine, ipratropium, and tiotropium); anti-virals (e.g. pyrimidine nucleosides including idoxuridine, and trifluridine; purine nucleosides including: vidarabine, and acyclovir; ribavirin, amantadine, interferon and its inducers, and other miscellaneous anti-virals, for example, thiosemicarbazones, zidovudine, and benzimidazoles); sympathomimetics (e.g., epinephrine); cardiovascular agents (e.g., calcium channel blockers: diltiazem, nifedipine, and verapamil); anti-arrhythmics (e.g., alprenolol, amiodarone, bretylium, diltiazem, flecainide, isoproterenol, lidocaine, metoprolol, nadolol, procainamide, propranolol, quinidine, timolol, and verapamil); vasoactive drugs (e.g., captopril, epinephrine, hydralazine, isoxsuprine, nitroglycerin, pentoxifylline, phentolamine, and prazosin); cardiotonics (e.g., dobutamine; dopamine; digitoxin; and digoxin); central nervous agents: e.g., anesthetics including barbiturates; anticonvulsants e.g., clonazepam, diphenylhydantoin, primidone; antidepressants: e.g., SSRI (selective serotonin re-uptake inhibitor); antiemetics: e.g., domperidone, metoclopramide; emetics: apomorphine; narcotic analgesics: codeine, demerol, fentanyl, hydrocodone, meperidine, morphine, oxymorphone, butorphanol, buprenorphine, pentazocine; non-narcotic analgesics including acetaminophen, aspirin, dipyrone; respiratory stimulants: e.g., caffeine, doxapram, zolazepam; sedatives/tranquilizers including: barbiturates; alpha 2 antagonists (e.g., detomidine, medetomidine, dexmedetomidine, carfentanil, diazepam, droperidol, ketamine, midazolam, phenothiazine tranquilizers (including acepromazine, chlorpromazine, ethylisobutrazine, promazine, and triflupromazine), romifidine, xylazine; diuretics (e.g., chlorothiazide, and furosemide); dental hygiene (e.g., glycerol monolaurate materials and orally active antibiotics); gastrointestinal agent (e.g., cimetidine (H2 agonist), famotidine, ranitidine, and omeprazole); hypotensives (e.g., acepromazine, and phenoxybenzamine); hormones (e.g., ACTH, altrenogest, estradiol 17beta, estrogens GNRH, FSH, LH, insulin, LHRH, megestrol, melatonin, misoprostol, norgestomet, progesterone, testosterone, thyroxine, and trenbolone); immunomodulators (stimulants including: levamisole, imiquimod and analogues, biological derivative products; and suppressants including: azathioprine); internal parasiticides (e.g., ivermectin, mebendazole, monensin, morantel, moxidectin, oxfendazole, piperazine, praziquantel, and thiabendazole); miotics (e.g. acetylcholine, carbachol, pilocarpine, physostigmine, isoflurophate, echothiophate, and pralidoxime); mydriatics (e.g., epinephrine, and phenylephrine); mydriatics/cycloplegics (e.g. atropine, scopolamine, cyclopentolate, tropicamide, and oxyphenonium); prostaglandins (e.g., cloprostenol, dinoprost tromethamine, fenprostalene, and fluprostenol); muscle relaxants (e.g., aminopentamide, chlorphenesin carbamate, methocarbamol, phenazopyridine, and tiletamine); smooth muscle stimulants (e.g., neostigmine, oxytocin, and propantheline); serotonin; urinary acidifiers (e.g., ammonium chloride, ascorbic acid, and methionine); and vitamins/minerals (e.g., Vitamins A, B, C, D, K, and E). The active agent may be encapsulated in various suitable forms, including solid forms, liquid forms, and the like. It will also be appreciated that one or more additional active agents could also be included in the particle, distributed throughout the polymer matrix (in addition to the active agent(s) in the emulsion droplet).

A protease inhibitor may be included in the dosage form contemplated herein as well. Examples of such protease inhibitors include, but are not limited to, AEBSF-HCI, Amastatin-HCI, (epsilon)-Aminocaproic acid, (alpha)1-Antichymotrypsin from human plasma, Antipain-HCL, Antithrombin III from human plasma, (alpha)1-Antitrypsin from human plasma, (4-Amidinophenyl-methane sulfonyl-fluoride), Aprotinin, Arphamenine A, Arphamenine B, Benzamidine-HCI, Bestatin-HCI, CA-074, CA-074-Me, Calpain Inhibitor I, Calpain Inhibitor II, Cathepsin Inhibitor Z-Phe-Giy-NHO-Bz-pMe, Chymostatin, DFP (Diisopropylfluoro-phosphate), Dipeptidylpeptidase IV InhibitorH-Giu-(NHO-Bz)Pyr, Diprotin A, E-64, E-64d (EST), Ebelactone A, Ebelactone B, EDTA-Na2, EGTA, Elastatinal, Hirudin, Leuhistin, Leupeptin-hemisulfate, (alpha)2-Macroglobulin from human plasma, 4-(2-Aminoethyl)-benzenesulfonyl fluoride hydrochloride, Pepstatin A, Phebestin, Phenyl methyl sulfonyl fluoride, Phosphoramidon, (1-Chloro-3-tosylamido-7-amino-2-heptanone HCI, (1-Chloro-3-tosylamido-4-phenyl-2-butanone), Trypsin inhibitor from egg white (Ovomucoid), and Trypsin inhibitor from soybean.

The particle used in the presently claimed and disclosed inventive concept(s) may also contain small quantities of butylated hydroxy toluene, glycerine, polyethylene glycols, propylene glycol, lecithin, antioxidants, tocopherol, docosahexaenoic acid, and pirotiodecane in addition to solubilizers, and extenders. Some examples of additional pharmaceutically acceptable additive include but are not limited to sugars, such as lactose, glucose and sucrose; starches such as corn starch and potato starch; powdered tragacanth; malt; gelatin; talc; excipients such as cocoa butter and suppository waxes; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; pyrogen-free water; isotonic saline; Ringer's solution, ethyl alcohol and phosphate buffer solutions, as well as other non-toxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

The amount of active ingredient that can be combined with the carrier materials to produce a single dosage form will vary depending upon the particular mode of administration.

Once the solid-in-oil-in-water emulsion is mixed with the polymer precursor and delivery-enhancing moiety, polymer droplets of the desired size can be generated to create the particles. In one aspect, the mixture of polymer/emulsion droplet mixture is extruded or dispensed from a suitable apparatus for forming droplets. Any suitable apparatus can be used, and will generally comprise a chamber for holding the polymer/emulsion droplet mixture, with the chamber being in fluid communication with a fluid passage that terminates in a dispensing outlet or tip. The dispensing tip will have an orifice through which the mixture is expelled as a precursor droplet (aka microdroplet). The technique can be executed using a simple apparatus, such as a syringe and needle, as well as machines specifically designed for droplet generation. A spray nozzle for generating several droplets simultaneously can also be used. Regardless, the desired size of the polymer/emulsion droplet can be controlled based upon the cross-sectional dimension of the orifice and the viscosity of the mixture, as well as by controlling the pressure used to generate the droplets (e.g., the pressure/energy of the gas in an ultrasonic nozzle), as will be appreciated by those in the art. Further, it will be appreciated that techniques for generating droplets of various sizes can vary depending upon the particular apparatus being used. In one aspect, the technique involves atomization of the mixture to generate delivery-enhanced droplets that can be crosslinked to yield the deliver-enhanced particles, such as by contacting the droplets with a suitable crosslinking agent, exposure to activating radiation or heat, or changing the pH, depending upon the selected polymer matrix. In one or more embodiments, the generated droplets are added dropwise to a solution of crosslinking agent, which in general will comprise an agitated or stirring bath of an effective amount of crosslinking agent dispersed in a solvent system for coacervation of the particles in the bath. In one or more embodiments, an effective amount of time-release excipient can also be included in the crosslinking bath. In some embodiments, the polymer/emulsion droplets are added to a bath containing the crosslinking agent (and optional time-release excipient) for coacervation and ionic gelation of the particles in the bath. In one or more embodiments, the generated polymer/emulsion droplets are rapidly frozen after generation, prior to initiating crosslinking. For example, as described in more detail below, the generated droplets can be contacted with liquid nitrogen to rapidly freeze the polymer suspension into droplet form, prior to crosslinking.

It will be appreciated that the particular crosslinking/curing conditions can be selected depending upon the particular polymer system selected for the matrix. It will be understood that the various reagents will be selected to minimize any adverse effects on the active agent. The resulting particles are individual/discrete self-sustaining bodies that generally comprise the crosslinked polymer matrix in a self-sustaining particulate form, with the emulsion droplets generally uniformly distributed throughout the particle. In one or more embodiments, a delivery enhancing moiety is continuously presented on the exterior surface of the crosslinked polymer matrix/particle regardless of any polymer degradation. Preferably, the delivery enhancing moieties are present on the external surface of the particle, and orientated such that their functional groups can interact with the GI's epithelial surfaces, enterocytes, and their receptors to enhance the mucoadhesive property of the particles and facilitate absorption of the particle's active agent.

In one or more embodiments, the polymer suspension comprises sodium alginate (or potassium alginate) as the polymer base building block which is then, in at least one embodiment, converted to a more stable (crosslinked) form through ionic exchange. Fabrication of alginate particles which are employed as delivery vehicles of therapeutics includes gelation and crosslinking of the alginate matrix. Gel formation of alginate materials is obtained either by lowering the pH or by inducing nucleation with a divalent cation such as calcium ions (Ca+2) which cross-links pairs of guluronic acid units within the alginate polymer structure. Divalent cations can be used for crosslinking of sodium or potassium alginate. Suitable divalent cations for use in the invention include any ions capable of forming a gel upon interaction with alginate. More particularly, the divalent cations are preferably biocompatible. In one or more embodiments, the divalent cations are selected from the group consisting of calcium, barium, strontium, and combinations thereof. Advantageously, the divalent cation reacts with the alginate to yield a microparticle for each droplet comprising an alginate matrix in which the active agent, delivery enhancing moieties, and optional time-release excipients, if present, are entrapped. For example, upon contacting sodium or potassium alginate droplets with an aqueous solution of calcium chloride (e.g., 1% to 40% by weight, preferably 1-24% or any effective concentration) or calcium acetate, for example, the sodium (or potassium) of the alginate droplet is replaced by calcium. This reaction occurs rapidly at room temperature (e.g., 20-25° C.) or below resulting in the formation of helix-helix loaded aggregates which rapidly separate from the aqueous medium in the form of a coacervated precipitate.

Regardless, the resulting particles encapsulating the active agent emulsion are preferably dried to a moisture (water) content ranging from <0.01-8% depending upon the active agent for encapsulation. The resulting particles can also be freeze-dried and/or lyophilized into a powder form for storage. The particles can be packaged in gel-caps, or pressed into tablets for suitable unit dosage forms.

Some embodiments of the inventive method involve rapid freezing of the liquid polymer/emulsion mixture droplets after droplet formation, but prior to crosslinking. For example, the methods include spraying a polymer suspension containing the emulsion droplets onto liquid nitrogen with a 120 kHz ultrasonic nozzle which produces frozen droplets (aka microdroplets) with the desired dimensions. It will be appreciated that embodiments can produce different sized droplets depending upon the desired product size and the capabilities of the nozzle(s) used.

The frozen droplets are then contacted with an appropriate crosslinking agent to crosslink the polymer matrix. For example, in the case of alginate, frozen alginate microdroplets are formed by spraying an alginate suspension containing the emulsion droplets, delivery enhancing moieties, and time-release excipients, if present, onto liquid nitrogen. The resulting frozen microdroplets are then collected (e.g., with a strainer, spoon, etc.) and transferred to a CaCl₂ bath, where they begin to crosslink as they thaw due to the cross-linking of the alginate polymer in the individual droplet with Ca⁺² ions thus forming crosslinked microparticles. In other words, the thawing and crosslinking process occurs simultaneously or near simultaneously, and proceeds from the outside-in as the Ca⁺² ions infiltrate the frozen alginate droplet during thawing. Coacervates are formed as a result of electrostatic interaction between two aqueous phases during thawing, followed by ionic gelation as the material transitions from liquid to gel due to ionic interaction conditions at room temperature.

As the microdroplets defrost and thaw, the surface of the thawing droplet comes in contact with the crosslinking agent (i.e., in the case of alginate, Ca⁺² ions) and as these crosslinking agents reach the polymer within the droplet, the gelation and crosslinking processes occur within each droplet, proceeding from the surface to the interior. The degree of coacervation is inversely related to cross-sectional dimensions (i.e., diameter) of the resulting product as intermolecular forces drive out water molecules. In one or more embodiments, the droplets remain in contact with the crosslinking agent for a period of time sufficient to completely cure/crosslink the entire droplet from the surface to the core. In one or more embodiments, the droplets are stirred in the crosslinking bath for a period of about 4 to about 6 hours. The inventive method prevents aggregation of droplets during coacervation, preventing the fusion of droplets that would increase the overall size or agglomeration of the particles.

After the particles are crosslinked, they are removed from the crosslinking bath by centrifugation and washed with an aqueous solution several times (e.g., water). Alternatively, the crosslinked microparticles can be washed with an aqueous solution containing additional time-release excipient in embodiments concerned with faster-release particles. The major advantages of the method are founded on obtaining uniform particle size distribution per batch prepared, including, but not limited to, batches of particles with a particle size, for example, of from about 3 μm to about 5 μm. Further, these particles contain substantially uniformly distributed emulsion droplets within each particle body. As used herein, references to the “particle size” refer to the maximum surface-to-surface dimension of a given particle, such as the diameter in the case of substantially spherical particles.

In one embodiment, the resulting crosslinked microparticles present a characteristic release of the emulsion droplets containing the active agent when these microparticles are exposed to the basic aqueous environment of the small intestine, which causes the swelling of the microparticles and allows the emulsion droplets containing the active agent to diffuse out of the polymer. Such microparticles are prepared according to the procedures described above, but without an accelerating time-release excipient. In an alternative embodiment, crosslinked microparticles present a characteristic faster release of an active agent when these microparticles are prepared from a suspension of polymer and emulsion droplets in the presence of an accelerating time-release excipient, such as mannitol. The disclosed methods are capable of producing particles with a particle size of greater than 1 μm, preferably from 1 μm to about 1000 μm, more preferably from 1 μm to about 200 μm, and even more preferably from about 1 μm to about 20 μm.

The resulting microparticles have a structure characterized by the polymer chains crosslinked into a 3-dimensional network (i.e., the polymer matrix), with a porous structure and open and closed cells, in which the emulsion droplets containing the active agent and other constituents can be entrapped. The polymer matrix is characterized by this semi-rigid network that is permeable to liquids and gases, but which exhibits no flow and retains its integrity in the steady state. In other words, the microparticles are each self-sustaining bodies. The term “self-sustaining body” means that the polymer matrix, once formed, retains its shape without an external support structure, and is not susceptible to deformation merely due to its own internal forces or weight. The self-sustaining body is not pliable, permanently deformable, or flowable, like a jelly, putty, or paste, but is resilient, such that the matrix body may temporarily yield or deform under force. In other words, the self-sustaining body will recoil or spring back into shape after minor compression and/or flexing—it being appreciated that the polymer matrix will crack, break, or shear under sufficient exertion of external pressure or force. The resulting polymer microparticles are thus, a matrix-type capsule, meaning that they hold the encapsulated material throughout the volume of the particle body, rather than having a distinct shell as in a core-shell type capsule.

As discussed herein, the microparticles are resistant to enteric degradation. The particle body will not significantly degrade in stomach acids, and will localize in the small intestine. Without wishing to be bound by theory, the microparticle temporarily adheres to the epithelial lining of the small intestine facilitated by the mucoadhesive properties of the polymer matrix and the delivery enhancing moieties, which are oriented on the particle surface such that their functional groups can interact with the epithelial lining. In one or more embodiments, exposure to the acidic pH environment of the stomach will initiate an ion exchange in the polymer matrix, weakening the 3-dimensional structure. In one or more embodiments, subsequent exposure to basic pH environment of the small intestine causes the microparticle to swell and expand as the chains of the polymer matrix repel one another. This “opening” of the polymer matrix increases its mucoadhesive properties and releases the emulsion droplets containing the active agent directly in the vicinity of the lining of the small intestine significantly increasing the efficiency and efficacy of the uptake of the active agent into the blood stream. The released emulsion droplets provide an additional layer of protection from degrading enzymes such as peptidases and proteases, increasing the dwell time of the active agent in the body before eventual degradation in the bloodstream. Thus, the entire system interacts with the enterocytes to facilitate transcellular transport of the active agent. In one or more embodiments, the delivery enhancing moieties further enhance this uptake process. The polymer matrix itself is then cleared and excreted through normal GI mechanisms.

In one or more embodiments, the emulsion droplets are dispersed in a carrier matrix, in lieu of the self-sustaining polymer particles. That is, instead of using the polymer matrix particles described above, the emulsion droplets can be delivered via another type of vehicle or carrier. Exemplary carrier matrices include various types of gel or liquid carrier compositions, such as hydrogels, liquid polymer systems, and the like, which can then be contained in a suitable dosage form (e.g., gel cap, etc.) for oral administration. The emulsion droplets can also themselves be directly encapsulated in a gel cap, capsule, and the like, or delivered via a solution or suspension (i.e., liquid unit dosage form). In some embodiments, small amounts of polymer (e.g., chitosan) may be included with emulsion droplets, either inside the emulsion droplet or as a crosslinked coating/shell around the individual droplets. The emulsion droplets can then be dispersed in the carrier matrix for administration.

In one or more embodiments, the emulsion droplets and/or microparticles are used for delivery of an active agent to a subject. In one or more embodiments, the emulsion droplets and/or microparticles can be used in a unit dosage form for oral administration of the active agent to a subject. The term “unit dosage form” refers to a physically discrete unit suitable as a unitary dosage for human or animal use. Each unit dosage form may contain a predetermined amount of the active agents in a carrier calculated to produce the desired effect.

In practice, the emulsion droplets and/or microparticles are administered to a subject in need thereof. Oral administration routes are particularly preferred according to embodiments of the invention; however, that does not necessarily preclude other modes of administration. In one or more embodiments, treatment methods comprise administering emulsion droplets and/or microparticles comprising a therapeutically-effective amount of active agent to the subject. As used herein, a “therapeutically effective” amount refers to the amount of the active agent that will elicit the biological or medical response of a tissue, system, animal, or human that is being sought by a researcher or clinician, and in particular elicit some desired therapeutic effect. One of skill in the art recognizes that an amount may be considered therapeutically effective even if the condition is not totally eradicated but improved partially. The microparticles are advantageously, resistant to gastric fluid (i.e., enterically resistant), but allows the particles to swell and release active agents at conditions consistent with the environment of the targeted region of the GI tract.

Depending upon the active agent and condition involved, a single dose may provide adequate therapeutic effect. In one or more embodiments, additional dosages can be administered, by the same or different route, to achieve the desired prophylactic or therapeutic effect. The emulsion droplets and/or microparticles can also be administered using a prime and boost regime if deemed necessary. In one or more embodiments, the emulsion droplets and/or microparticles are administered to the subject as part of ongoing treatment, such as in the case of a daily treatment regimen.

Regardless of the embodiment, once administered, the microparticles preferably localize in the GI tract, temporarily adhere to the intraluminal surface of the lining of the GI tract, release their active agent payload which enters the blood stream, and then the remainder of the particles themselves are excreted. In other words, the microparticles according to the various embodiments of the invention do not themselves cross the intestinal mucosa, and are not capable of transmucosal passage across the intestinal mucosa into the intestinal bloodstream. Rather, the polymer matrix is degraded and/or swelled in the GI tract, releasing the emulsion droplets containing the active agent, which is selectively taken up into the intestinal bloodstream (without the rest of the particle).

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

### Preparation of Sodium Alginate Microparticles Containing an Insulin Microemulsion

Multiphase emulsions were generated in a multi-step process creating a primary emulsion consisting of insulin, beta-carotene, and oil with a surfactant and then suspending these primary emulsions in an aqueous mixture of alginate modified with the addition of a targeting molecule, resulting in a second, higher order, suspension.

A formulation of zinc-insulin was prepared by adding insulin in HCl and ZnCl₂ in the presence of m-cresol and glycerin. A 0.1N HCl solution was prepared by combining 100 mL of HPLC-grade water with 83 μL HCL and thoroughly mixing at room temperature. In a separate container, a 0.1N NaOH solution was prepared by combining 20 mL of HPLC-grade water with 80 mg of NaOH, followed by mixing on a stir plate at room temperature until completely dissolved. A solution of ZnCl₂ was prepared by mixing the desired amount of ZnCl₂ to 10 mL of HPLC-grade water, followed by mixing well a room temperature. Approximately 0.034 mg Zn is used per 100 IU Insulin.

The formulation was prepared in a 50 mL beaker on a stir plate at room temperature. First, 40 mL of 0.1N HCl was added to the beaker, followed by 3.192 mL of ZnCl₂ solution using a pipette. Next, 0.180 mL m-Cresol (3.7 mg/ml) was added to the beaker using a pipette, followed by 0.960 mL Glycerin (24 mg/ml). This mixture was stirred for 5 minutes at room temperature (Stirrer speed: 1). Next, approximately 750 mg Insulin powder (Sigma Aldrich; solubility insulin 20 mg/ml in 0.01N HCl) was added to this solution, followed by stirring until completely dissolved. In some cases, the pH of the solution can be checked before addition of glycerin and adjusted if needed with HCl or NaOH to a pH of 7.0. In this example, the actual quantity of insulin used was calculated to be 730.6 mg.

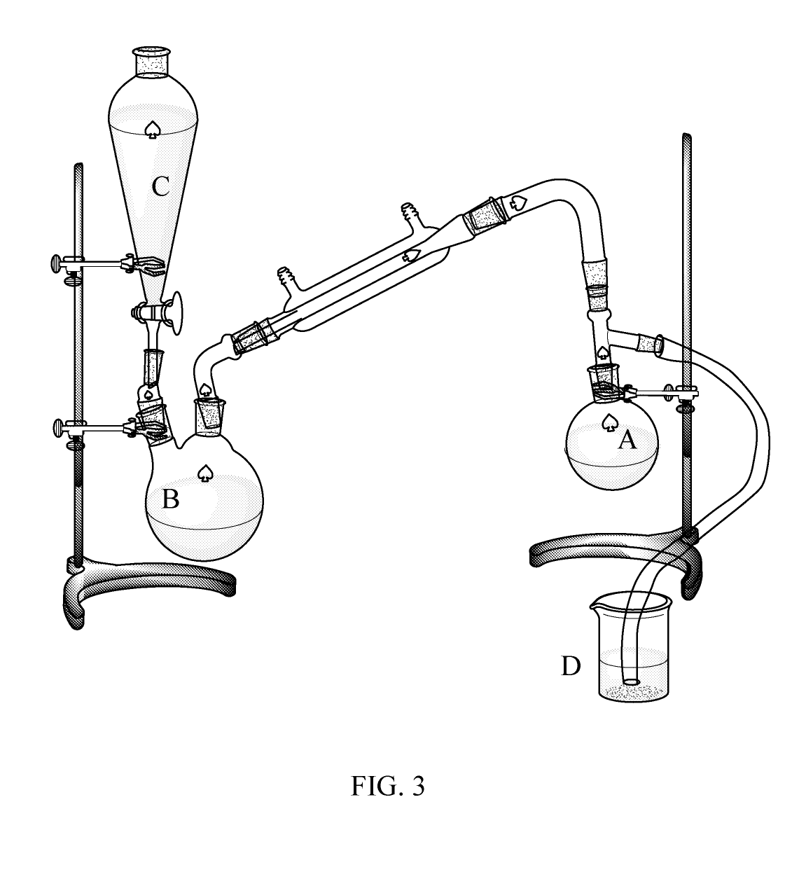

Preparation of the BCC was carried out using the apparatus shown in FIG. 3. First, 5 g LycoRed Beta-carotene (Lyc-O-Beta 30% Suspension in Sunflower Oil, lot # BCS-30-011602) was combined with 100 mL acetone solution in a reaction vessel (A). Next, 3 g NaCl were placed in a double neck round bottom flask (B), and an amount of sulfuric acid was placed into separatory funnel (C) positioned above the round bottom double neck flask as shown. Together, B and C serve as an HCl gas generator.

The separation funnel stopcock was opened to allow dropwise addition of sulfuric acid into the NaCl in the flask. This generated a steady stream of HCl gas to flow directly into the reaction vessel containing the beta-carotene (FIG. 3, A). Gas exposure was continued at room temperature for approximately 30 minutes, after which time the HCl generation was stopped by closing the stopcock of the separation funnel and the single neck flask (A) was detached from the assembly, oxygenating the reaction product upon exposure to ambient air. The resulting chlorination product of the beta carotene (referred to herein as beta-chloro-carotene or “BCC”) was then separated via distillation.

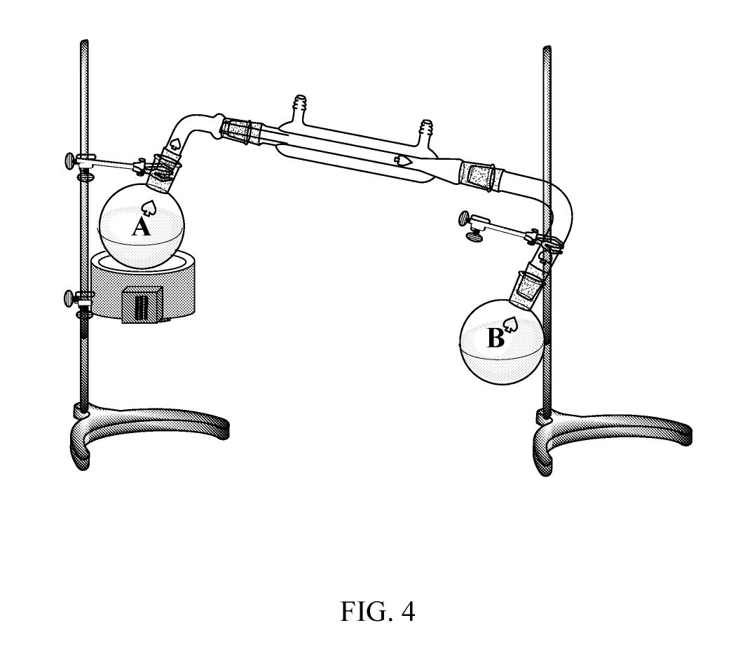

The reaction vessel (A) was attached to a distillation apparatus (FIG. 4), covered with aluminum foil (not shown), and heated to begin distillation to remove the acetone from the resulting. The distilled acetone was collected in a flask. After distillation was complete, the reaction vessel was allowed to cool to room temperature. The resulting BCC was transferred to a 200 mL bottle for storage at 4° C. until use.

The Zinc-Insulin Complex Formulation was centrifuged at 9,000 rpm for 15 min at 4° C., followed by removal of the supernatant to yield an insulin precipitate. The supernatant was collected for later use. The primary emulsion was prepared by dissolving 200 mg beta-carotene (LycoRed) in 50 g of sunflower oil. Next, 6 ml of the beta-carotene/oil mixture was added to 720 mg of Span 80 to yield the primary emulsion. The primary emulsion was homogenized in an ice bath for 30 seconds (level 6) before addition of the insulin precipitate, and homogenization for an additional 1 min in an ice bath. The resulting S/O emulsion was then added to chilled Tween-80 (100 mg in 100 mL HPLC-grade water), followed by homogenization for 3 minutes to stabilize the emulsion particles and yield a solid-in-oil-in-water (S/O/W) multiple emulsion.

The S/O/W multiple emulsion was added to a high pressure homogenizer and homogenized at approximately 10,000 psi for three passes. This emulsion can alternatively be prepared by ultrasonic probe sonication.

In order to prepare the sodium alginate (NaAA) gel with insulin emulsion, the S/O/W emulsion was mixed with 17 mg sodium alginate and mixed with a spatula to form a paste. The supernatant collected above was added, followed by the addition of HPLC-grade water (for a total quantity of 300 mL). Next, the prepared BCC was added to the alginate mixture, and mixed thoroughly.

Microparticles were generated using a 5% CaCl₂) solution prepared by mixing 25 g CaCl₂) with 500 mL HPLC-grade water, followed by filtering through a 0.2 μm Nalgene filter. A bath of this solution was created for the crosslinking step.

A Buchi spray nozzle was used to aerosolize the BCC-NaAA gel formulation at a rate of 3.0 mL/min. under 5 psi N₂ gas. In particular, 45 mL of the BCC-NaAA formulation was loaded into a syringe and connected to a Harvard syringe pump. Droplets were sprayed into the CaCl₂) bath for crosslinking. The resulting particles were allowed to gel in the CaCl₂) solution for 30 minutes and then filtered, with the filtrate being reserved for analysis. The resulting particles were washed three times with HPLC-grade water and transferred to petri dishes where they were frozen with liquid nitrogen and finally lyophilized for 48 hours.

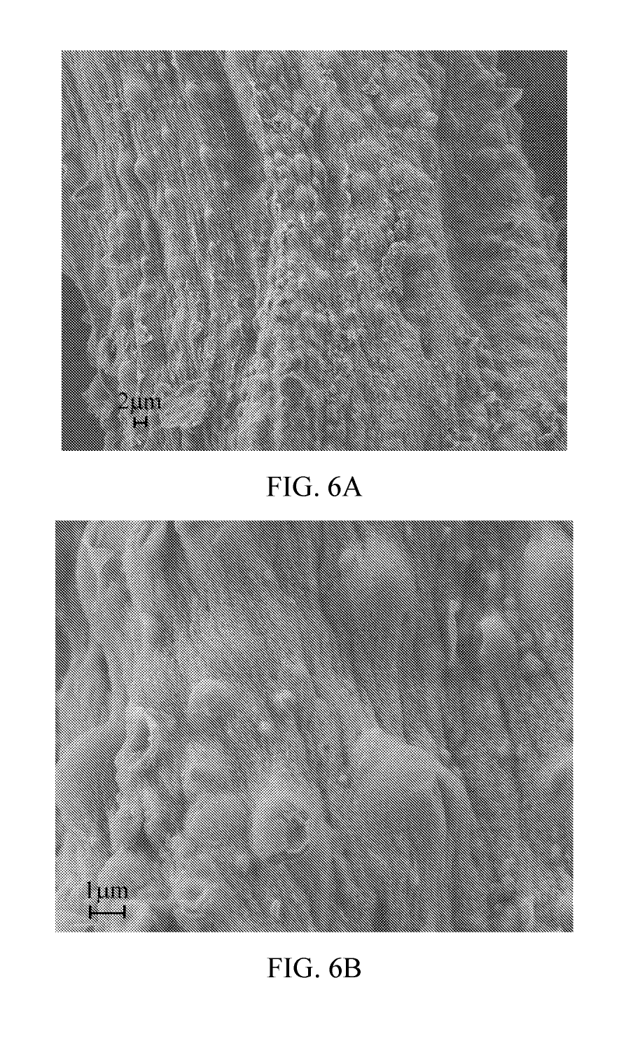

The resulting spherical particles are illustrated in representative form in FIG. 5A. An SEM image shows a representative particle in FIG. 5B (particle size of about 30-32 μm shown). FIGS. 6A and 6B show the inside of a particle, where the emulsion droplets can be seen dispersed in the particle matrix.

### Example 2

### Testing of Microemulsion Particles for Total Insulin Concentration

Total insulin amounts incorporated into the final emulsions were measured by elution from the preparations described above. Three different insulin-containing microemulsion particles were initially generated using the basic protocol from Example 1. The formulations are defined in Table 1 below. Formulations TA-3 and TA-4 were made with emulsions of zinc-complexed insulin. These formulations differed in the addition of BCC, where TA-3 included BCC, but TA-4 did not. Formulation TA-7 included BCC, but was made with uncomplexed insulin powder dissolved in 0.01N HCl without m-cresol, zinc, or glycerin.

Following the preparation of each of the described formulations, the amount of insulin contained in each formulation was assayed by eluting insulin with NaOH, which serves to swell the particles and allow entrapped active agents to diffuse out of the particle and into the surrounding media. The amount of insulin eluted in this way was measured by HPLC and is presented below as the Average Experimental Dose (IU/kg).

TABLE 1
Insulin Emulsion Formulations

| | | Average |
|---|---|---|
| Formulation | | Experimental |
| Name | Description | Dose (IU/kg) |
| TA-3 | Emulsion with BCC and Zinc | 54 |
| TA-4 | Emulsion with Zinc | 53 |
| TA-7 | Emulsion BCC | 54 |

### Example 3

### In Vivo Testing of Microemulsion Particles in a Rodent Model

The Pharmacokinetics of the insulin-containing emulsion particles generated in Table 1 were assayed in 10 week old Sprague-Dawley(SD) rats (Charles River Laboratories) treated with streptozotocin (STZ) to eliminate pancreatic islet β-cells and induce a widely used type I diabetes model. Animals were monitored for blood glucose levels daily over 10 days using a handheld glucometer to verify the disruption of insulin production. Animals exhibiting demonstrated hyperglycemia were selected for each group. Prior to the experimental procedure, animals were weighed and randomized into groups based on body weight. All rats were then fasted for five hours prior to dosing.

In the absence of pancreatic β-islet cells, these animals produce no endogenous insulin and all insulin can be attributed to the active agent of the compound. The formulations described in Example 2, Table 1 were used to establish the bioavailability of orally administered insulin from each of the emulsions. Control animals were given 5 IU/kg s.c. or 2.5 IU/kg i.v., while experimental animals were dosed orally with 50 IU/kg of microemulsion particles. Food was returned immediately post-dosage and blood was collected at the indicated time points (15, 30, 30, 60, 90, 120, 150, 180, 210, 240, 270, 300, 480, 720, and 1440) over a 24 hr. period. Whole blood was processed to plasma and analyzed for insulin quantity.

Briefly, relative bioavailability is a way of determining how much of an active agent gets into circulation following any variety of administrations routes. These measurements normalize against subcutaneous injections. Parameters were estimated using Watson pharmacokinetic software 7.4.2 (Thermo Electron Corporation) using a non-compartmental approach consistent with the various routes of administration. All parameters were generated from Insulin individual animal concentrations in plasma. Parameters were estimated using nominal sampling times to be relative to the start of each dose administration (within an acceptable tolerance limit). The average experimental dose for active ingredient, as per the study protocol, was used for dose-normalization purposes, 5.0 IU/kg, 2.5 IU/kg and 50 IU/kg for s.c. insulin, i.v. insulin, and the experimental groups respectively.

The area under the test item concentration versus time curve (AUC) was calculated using the linear-log linear trapezoidal method. AUC was not calculated for PK profiles with less than 3 consecutive quantifiable concentrations of test article at separate time points. When practical, the terminal elimination phase of each concentration versus time curve was identified using at least the final three observed concentration values. The slope of the terminal elimination phase was determined using linear regression on the unweighted concentration data. Parameters relying on the determination of the terminal elimination phase were excluded from the summary statistics where the extrapolation of the AUC to infinity represented more than 40% of the total area and/or if the number of regression points were less than three. The parameters described below were reported to 3 significant figures.

Mean insulin and glucose concentrations from the blood of animals were assessed following dosing of 5 IU/kg subcutaneous insulin, 50 IU/kg per os (p.o., i.e., orally) administered TA-3 (BCC with zinc), 50 IU/kg p.o. administered TA-4 (zinc without BCC), and 50 IU/kg p.o. administered TA-7 (BCC without zinc).

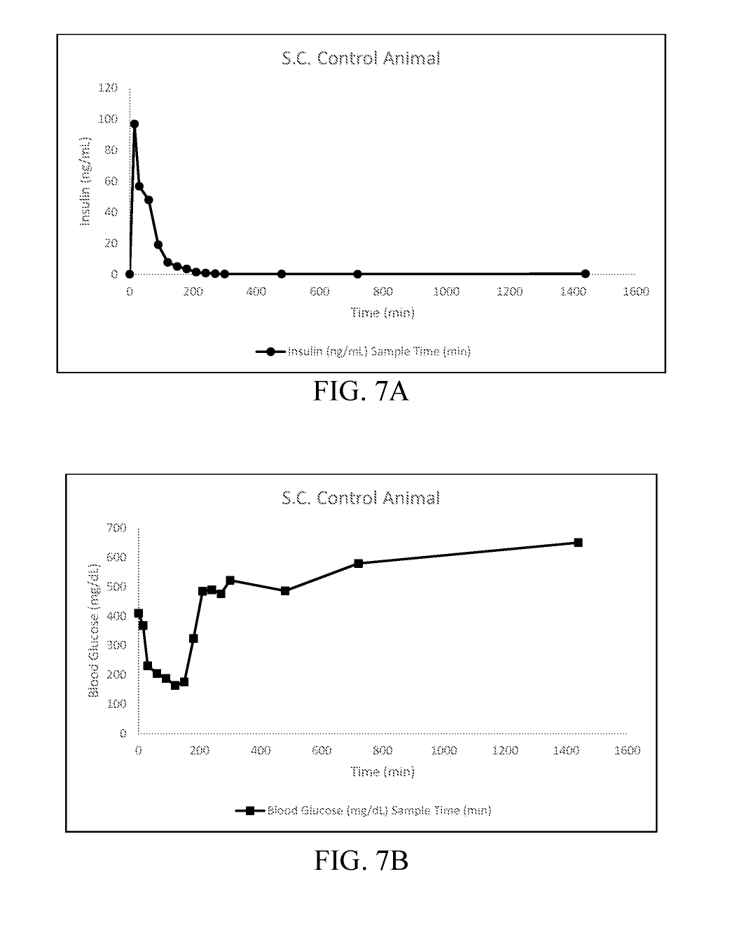

As a model of the current mode of insulin administration, control animals received an s.c. administration of insulin. Blood samples were taken over the next 24 hours and assayed at various time points to establish a reference curve for plasma concentration. These control animals' blood insulin levels are presented in FIG. 7A, blood glucose is presented in FIG. 7B together, these data illustrate the rapid appearance and peak of insulin in the blood as is typical following s.c. administration and corresponding declines in blood glucose.

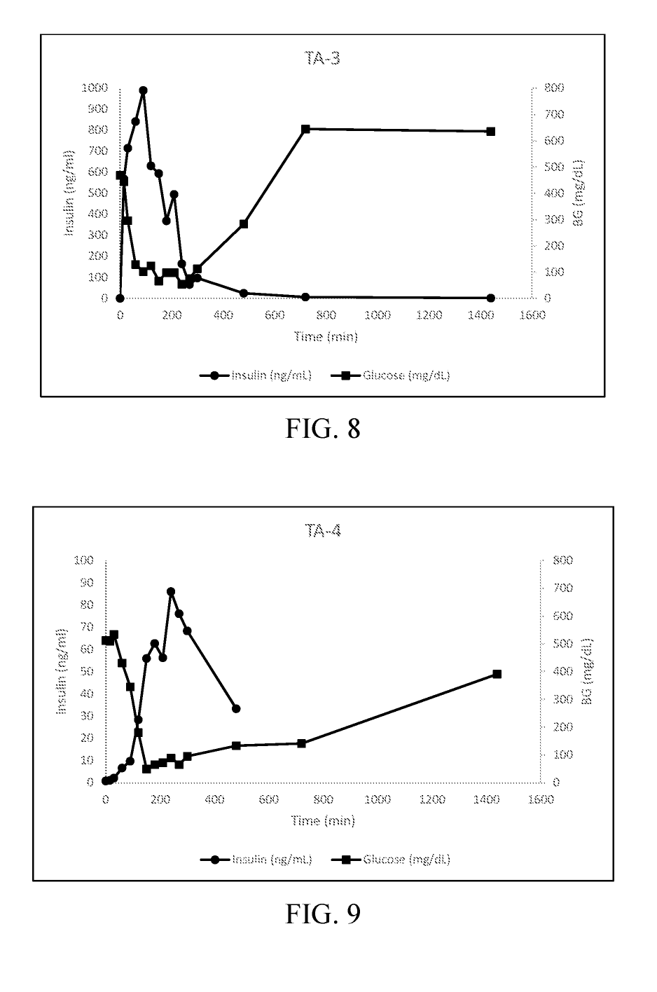

Three formulations of emulsion filled particles were dosed p.o. as indicated above. Concentrations of insulin over time are illustrated for each formulation from which exemplary data are shown. FIG. 8 shows blood insulin and glucose concentrations from an exemplary animal following administration of zinc-complexed insulin in emulsion with BCC (TA-3) at 50 IU/kg p.o. Compared to the control (s.c. injected) animals, Tmax of the absorbed blood insulin level and its corresponding pharmacodynamic effects are time-shifted to the right.

The administration of zinc-complexed insulin in the emulsion particles without BCC (TA-4) at 50 IU/kg p.o. was also carried out. FIG. 9 shows the resulting blood insulin and glucose concentrations in an exemplary animal following dosage.

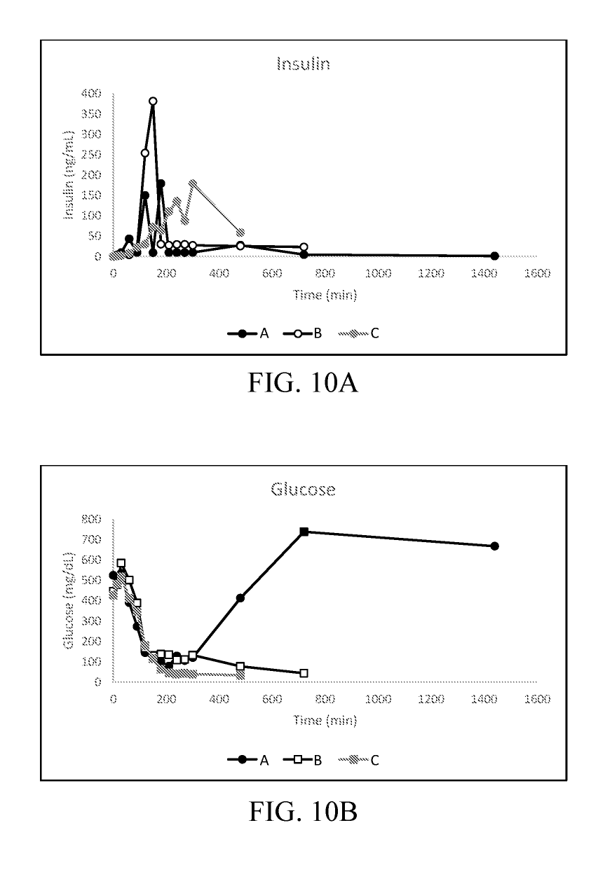

FIG. 10A shows blood insulin concentrations following administration of uncomplexed insulin in emulsion with BCC (TA-7) at 50 IU/kg p.o. for three animals in this group. FIG. 10B shows blood glucose levels.

All groups resulted in the detection of insulin in the blood of some animals peaking after administration either immediately or with some delay between 30 minutes up to 3 hrs post-dosing. In these tests, the zinc-complexed insulin in emulsion with BCC showed the highest peak concentration of insulin in the shortest time period post-dosing. Uncomplexed insulin in emulsion with BCC also effectively delivered insulin into the blood, as did the zinc-complexed insulin without BCC targeting (but in lower concentrations). As a group, the blood insulin concentration and respective pharmacodynamic response curves show both a time-shifted Tmax and indication of an extended absorption profile over that of the subcutaneous insulin injected control. Additionally, the data from TA-3 and TA-4 may indicate the absorbed insulin is directly entering the portal vein and is being absorbed by the liver and effecting blood glucose levels prior to being detected in the blood (hepatic effect).

Table 2 presents aggregated relative bioavailability data for exemplary animals given administrations of the formulations described in Table 1. The TA-3 formulation of zinc-complexed insulin in alginate with BCC resulted in the highest relative bioavailability of insulin at 258%.

TABLE 2
Relative Bioavailibility of Insulin

| PK | | | | | | |
|---|---|---|---|---|---|---|
| Parameter | Formulae | TA-3 | TA-4 | TA-7-A | TA-7-B | TA-7-C |
| AUC | AUC test/AUC ref | 25.8 | 4.08 | 3.76 | 6.24 | 7.33 |
| Dose | Dose ref/Dose test | 0.1 | 0.1 | 0.1 | 0.1 | 0.1 |
| t1/2 | t_{1/2} ref/_{t1/2}test | 0.157 | 0.225 | 0.167 | 0.0225 | 0.352 |
| AUC Extrap | AUC_{(0-∞)} test/AUC_{(0-∞)} ref | 25.8 | 5.67 | 3.82 | 17.6 | 9.12 |
| F Method 1 | Dose * AUC | 2.58 | 0.408 | 0.376 | 0.624 | 0.733 |
| F Method 2 | Dose * AUC * t_{1/2} | 0.405 | 0.0918 | 0.0628 | 0.014 | 0.258 |
| F Method 3 | Dose * AUC_{(0-∞)} | 2.58 | 0.567 | 0.382 | 1.76 | 0.912 |
| F Method 4 | Dose * AUC_{(0-∞)}* t_{1/2} | 0.405 | 0.128 | 0.0638 | 0.0396 | 0.321 |
| F Method 5 | Dose * (AUC test/AUC_{(0-∞)} ref) | 2.57 | 0.406 | 0.374 | 0.622 | 0.73 |

Percent Relative Bioavailability of Insulin

| Relative | | | | | | |
|---|---|---|---|---|---|---|
| Bioavailability | Formulae | TA-3 | TA-4 | TA-7-A | TA-7-B | TA-7-C |
| F₍₀₋ₓ₎ | (%) F Method 1 | 258 | 40.8 | 37.6 | 62.4 | 73.3 |
| F_{(0-∞)} | (%) F Method 3 | 258 | 56.7 | 38.2 | 176 | 91.2 |

Reference - s.c. PK parameters

### Example 4

### Testing of Microemulsion/nanoemulsion Particles for Total Insulin Concentration

Three different insulin-containing microemulsion particles were generated using the basic protocol from Example 1, except that the overall particle size was reduced, with the average particle size ranging from about 100 to about 500 μm in diameter. The formulations are defined in Table 1 below. Formulation TA-1 was a repeat of TA-3 from example 2 and 3 above. Formulations TA-6 was made with nano-sized emulsion particles. Formulation TA-7 (which differs from TA-7 in examples 2-3) was a repeat of formulation TA-3 from examples 2 and 3 above, though animals given this formulation were also given a high fat diet of cream cheese to study its effects on absorption. For comparison purposes, formulation TA-5 was made without emulsion particles (i.e. insulin was dispersed within the alginate matrix). Unlike example 3 where the Average Experimental Dose target was ˜54 IU/kg, in this example the Average Experimental Dose was reduced to ˜33 IU/kg.

Following the preparation of each of the described formulations, the amount of insulin contained in each formulation was assayed by eluting insulin with NaOH, which serves to swell the particles and allow entrapped active agents to diffuse out of the particle and into the surrounding media. The amount of insulin eluted in this way was measured by HPLC and is presented below as the Average Experimental Dose (IU/kg).

TABLE 3
Insulin Emulsion Formulations

| | | Average |
|---|---|---|
| | | Experimental |
| ID No. | Definition | Dose (IU/kg) |
| TA-1 | Microemulsion BCC/alginate particles with zinc- | 32 |
| | complexed insulin and BC | |
| TA-5 | No emulsion, zinc-complexed insulin dispersed | 32 |
| | in alginate matrix with BCC | |
| TA-6 | Nanoemulsion BCC/alginate particles with zinc- | 33 |
| | complexed insulin and BC | |
| TA-7* | Microemulsion BCC/alginate particles with zinc- | 33 |
| | complexed insulin and BC, concurrently fed a | |
| | high fat diet | |

### Example 5

### In Vivo Testing of Additional Insulin-Containing Microemulsion Particles in Rodent Model

A second study was performed as described in example 3 to assess the pharmacokinetics of a second batch of insulin-containing emulsion particles generated according to the definitions in Table 3, which vary according to emulsion particle (size and presence) and the availability of a high fat diet following administration of the compound. As above, experiments were assayed in 10 week old Sprague-Dawley(SD) rats (Charles River Laboratories) treated with streptozotocin (STZ) to eliminate pancreatic islet β-cells and induce a widely used type I diabetes animal model. The animals were monitored for blood glucose levels daily over 10 days using a handheld glucometer to verify the disruption of insulin production. Animals exhibiting demonstrated hyperglycemia were selected for each group. Prior to the experimental procedure, animals were weighed and randomized into groups based on body weight. All rats were then fasted for five hours before dosing. The group given TA-1 had food withheld for an additional 4 hours while groups given TA-5, 6, and 7 had food immediately returned following dosing. Groups TA-5 and TA-6 were given Purina Rat Chow while TA-7 was given cream cheese. All the emulsion particles contained BC in the emulsion and BCC in the alginate matrix. TA-5 had BCC in the alginate matrix.

The three emulsion formulations and one non-emulsion formulation were dosed p.o. as indicated above, as compared to a control s.c. administration of 5 IU/kg. Concentrations of insulin over time are illustrated for each formulation from which exemplary data are shown.

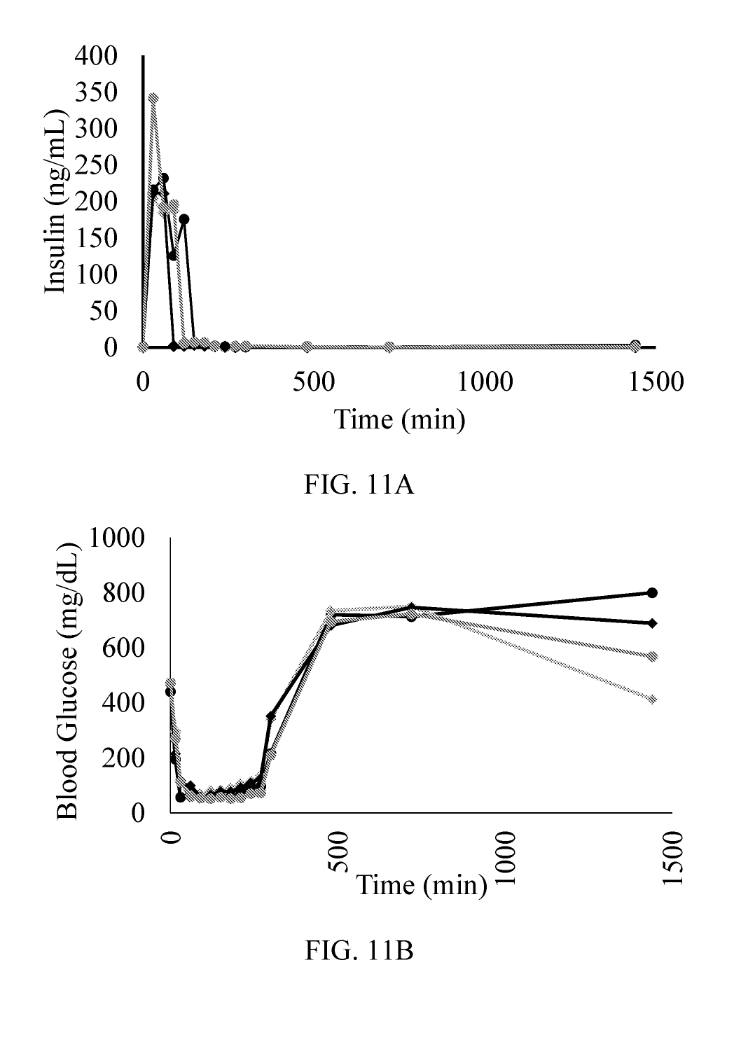

FIG. 11A illustrates the blood insulin following control s.c. injections of 5 IU/kg unencapsulated insulin. FIG. 11B, shows the resulting drop in blood glucose concentrations beginning just as insulin is detected in the blood.

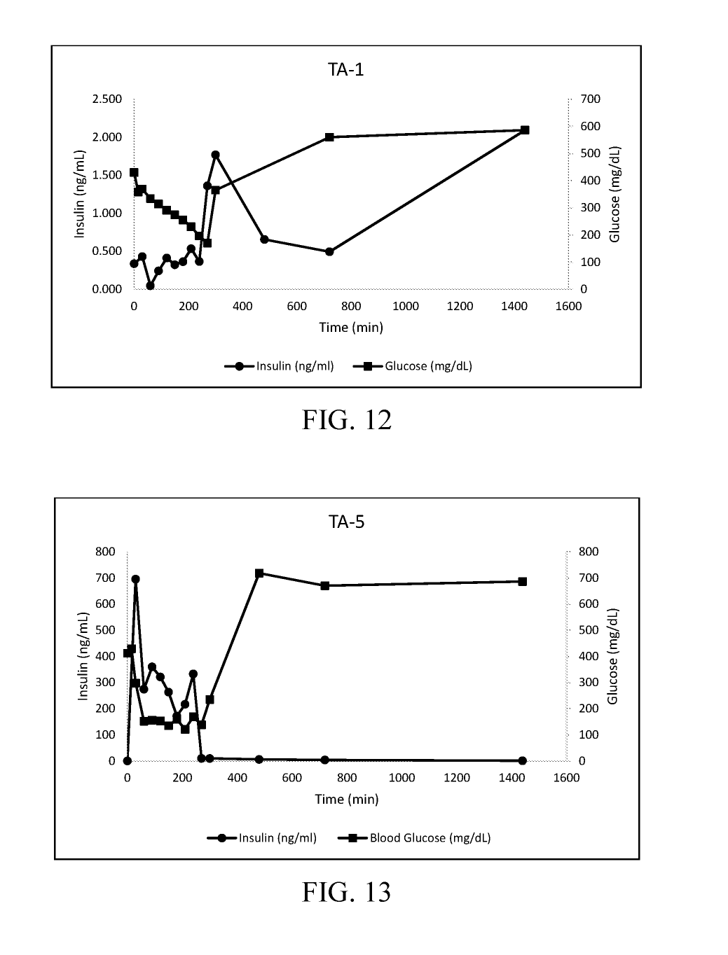

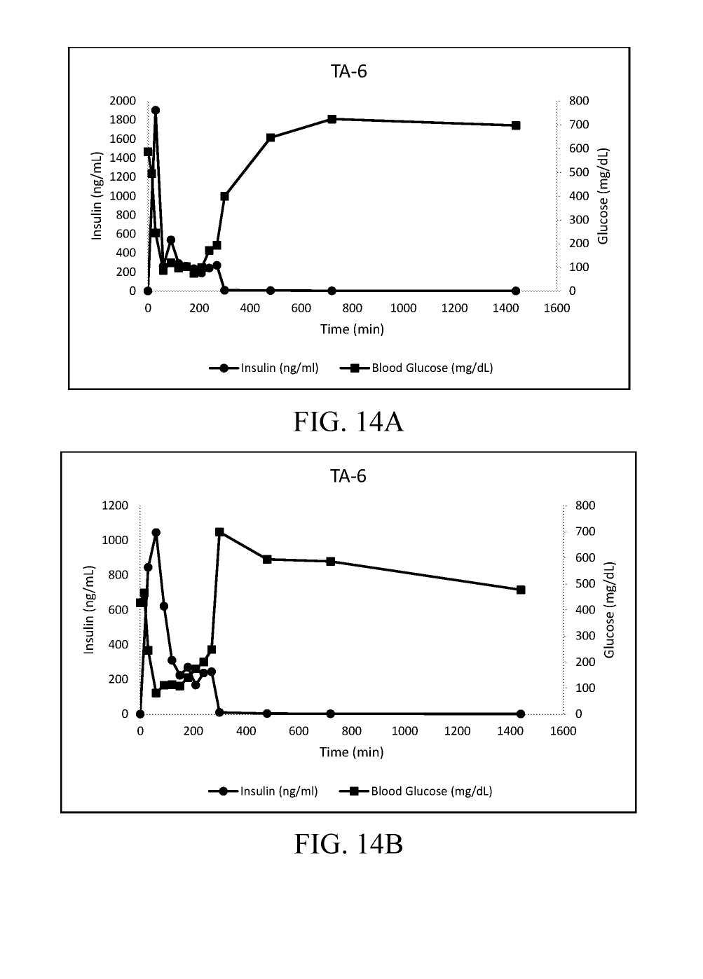

FIG. 12 illustrates the blood insulin and blood glucose concentrations for an exemplary animal administered the microemulsion particles with zinc-complexed insulin (TA-1). FIG. 13 illustrates the blood insulin and blood glucose concentrations for an exemplary animal administered zinc-complexed insulin dispersed in alginate matrix (without emulsion, TA-5). FIGS. 14A-B illustrate the blood insulin and blood glucose concentrations for two animals in the study administered nanoemulsion particles with zinc-complexed insulin (TA-6). These figures demonstrate the absorption of insulin and the resulting drop in blood glucose for the different formulations.

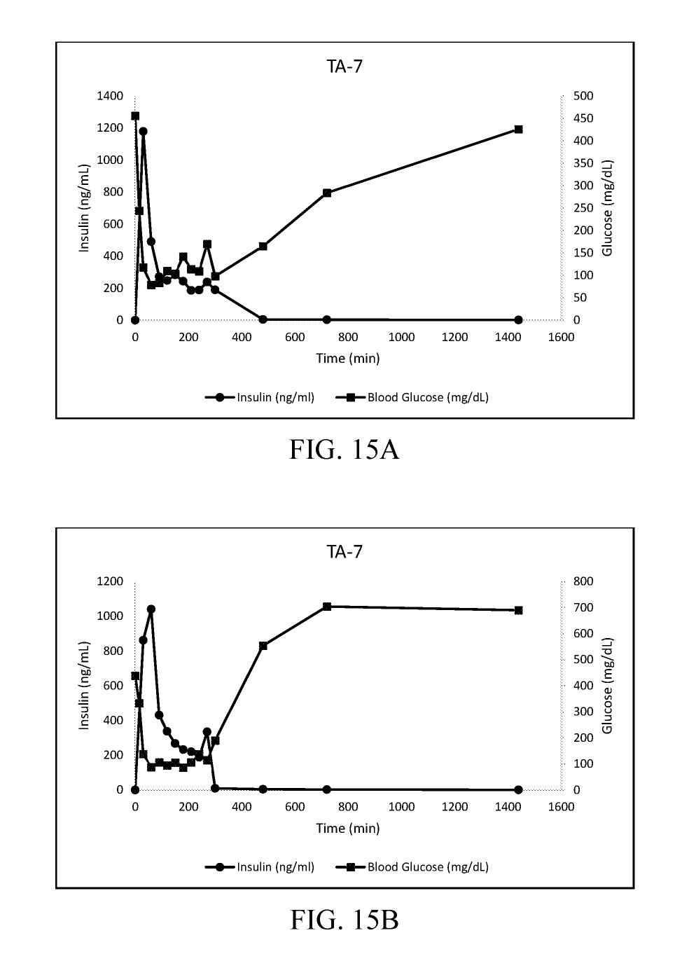

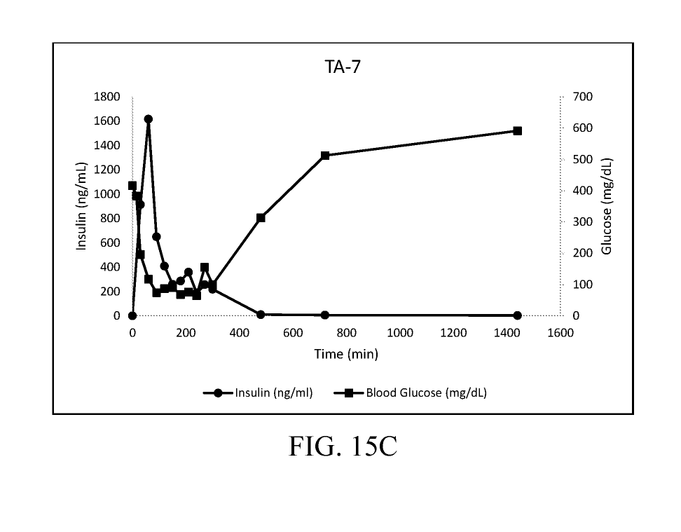

In order to assess the effect of dietary fat in the uptake of the compounds, animals dosed with TA-7 were allowed immediate access to high fat food after administration. FIGS. 15A, B and C show blood insulin concentrations following administration of insulin in emulsion with BCC (TA-7) at 33 IU/kg p.o. and with immediate access to a high fat meal for three different animals. Again, insulin peaks rapidly followed by disposal of blood glucose from circulation. The presence of dietary fat immediately after administration appears to have had an effect on the uptake/bioavailability (See Table 4) of insulin in animals given the inventive formulations.

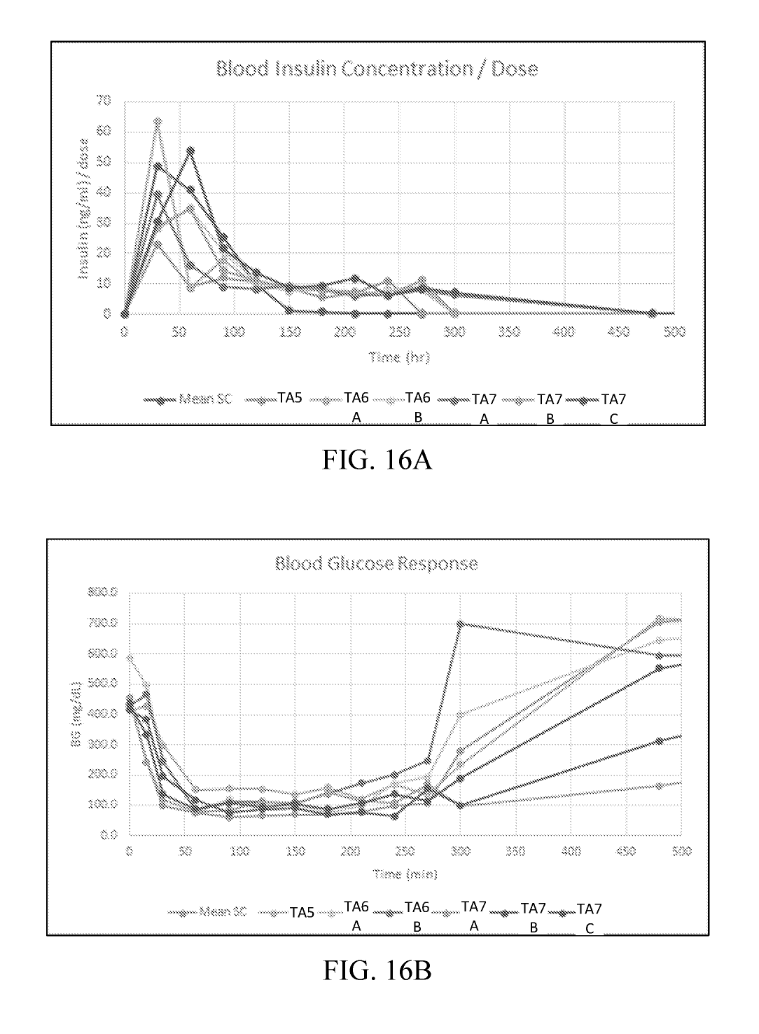

FIGS. 16A-16B show collective data for blood insulin and blood glucose levels for several animals in the study, in comparison to s.c. administration of insulin. The data demonstrates that the emulsion particles can be used to deliver insulin orally with effective outcomes that are comparable to s.c. administration of insulin.

Table 4 shows the aggregated summary of assays demonstrating the relative bioavailability of insulin from three exemplary animals given the TA-7 formulation described above.

TABLE 4
Individual TA-7Animals

| PK Parameter | Formulae | TA-7 A | TA-7 B | TA-7 C |
|---|---|---|---|---|
| AUC | AUC test/AUC ref | 21.7 | 22.4 | 31.6 |
| Dose | Dose ref/Dose test | 0.167 | 0.167 | 0.167 |
| t½ | t_{1/2} ref/_{t1/2} test | 0.0639 | 0.142 | 0.0846 |
| AUC Extrap | AUC_{(0-∞)} test/AUC_{(0-∞)} ref | 21.9 | 22.3 | 31.7 |
| F Method 1 | Dose * AUC | 3.62 | 3.73 | 5.27 |
| F Method 2 | Dose * AUC * t_{1/2} | 0.231 | 0.530 | 0.446 |
| F Method 3 | Dose * AUC_{(0-∞)} | 3.65 | 3.72 | 5.28 |
| F Method 4 | Dose * AUC_{(0-∞)} * t_{1/2} | 0.233 | 0.528 | 0.447 |
| F Method 5 | Dose * | 3.60 | 3.71 | 5.24 |
| | (AUC test/AUC_{(0-∞)} ref) | | | |

Percent Relevant Bioavailability of Insulin in TA-7 animals

| Relative | | | | |
|---|---|---|---|---|
| Bioavailability | Formulae | TA-7 A | TA-7 B | TA-7 C |
| F₍₀₋ₓ₎ | (%) F Method 1 | 362 | 373 | 527 |
| F_{(0-∞)} | (%) F Method 3 | 365 | 372 | 528 |

Reference - SC PK parameters

Altogether, these data indicate that insulin can be effectively protected and delivered into the blood of patients in bioactive forms following oral administration of the inventive compounds. This represents an important advance in the ability to administer protein drugs in innovative ways orally and without digestive inactivation. The presence of specific targeting molecules may assist with effective entry of the active ingredient into circulation, as well as appropriate activity of that agent once in circulation. Adjusting the inventive emulsion particle formulations additionally allows for novel pharmacokinetics and pharmacodynamics of the administered proteins.

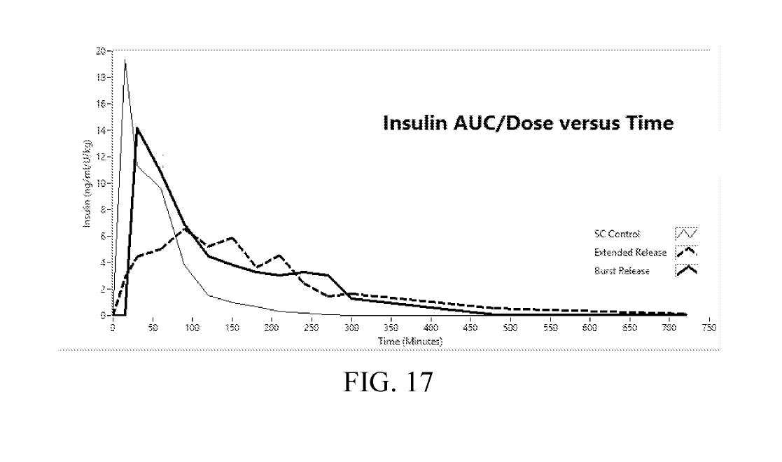

Example 5 differed from Example 3 in that we adjusted particle size and observed a different average elution profile. Example 3 showed a right shifted Tmax with extended absorption compared to control and Example 5 has an average profile closer to the control profile. FIG. 17 shows a comparison of Example 3 to Example 5 in comparison to an s.c. control.

1. A composite microparticle for oral delivery of an active agent to a subject, said composite microparticle comprising a self-sustaining body having an exterior surface, said self-sustaining body comprising a crosslinked polymer matrix and a plurality of emulsion droplets distributed throughout said polymer matrix, said active agent being encapsulated in said emulsion droplets, said composite microparticle further comprising a plurality of delivery enhancing moieties, wherein at least a portion of said delivery enhancing moieties are presented on the exterior surface of said self-sustaining body and/or on said emulsion droplets, wherein said microparticle is resistant to enteric degradation and will localize in the gastrointestinal tract of said subject without crossing the intestinal mucosa into the intestinal bloodstream.

2. (canceled)

3. The composite microparticle of claim 1, wherein said emulsion droplets comprise said active agent, an oil, a surfactant, and optional delivery enhancing moieties encapsulated therein.

4. The composite microparticle of claim 1, wherein said emulsion droplets comprise an active agent in a solid-in-oil-in-water emulsion.

5. The composite microparticle of claim 1, wherein said delivery enhancing moieties are ionically, covalently, or supramolecularly bound to the polymer matrix, physically entrapped within the polymer matrix, and/or embedded within the emulsion droplets.

6.-8. (canceled)

9. The composite microparticle of claim 1, said microparticle further comprising one or more time-release excipients distributed throughout said polymer matrix.

10.-16. (canceled)

17. The composite microparticle of claim 1, wherein at least a portion of said delivery enhancing moieties are presented on the exterior surface of both of said self-sustaining body and on said emulsion droplets.

18. The composite microparticle of claim 17, wherein said delivery enhancing moieties on said self-sustaining body are different from said delivery enhancing moieties on said emulsion droplets.

19. A composition comprising a therapeutically-effective amount of a plurality of composite microparticles according to claim 1 dispersed in a pharmaceutically acceptable carrier.

20.-22. (canceled)

23. A method of orally administering an active agent to a subject in need thereof, said method comprising orally administering a therapeutically effective amount of microparticles according to claim 1 to said subject.

24. (canceled)

25. A method of forming a composite microparticle, said method comprising:
combining a polymer suspension with an active agent emulsion to yield a mixture, wherein said polymer suspension comprises a polymer matrix precursor dispersed in a solvent system, and wherein said active agent emulsion comprises an active agent encapsulated in an emulsion, and wherein said polymer suspension and/or said active agent emulsion further comprise a plurality of delivery enhancing moieties; and
crosslinking said polymer matrix precursor in said mixture to yield a crosslinked polymer matrix in the form of a self-sustaining body having an exterior surface and a plurality of emulsion droplets comprising said active agent distributed throughout said polymer matrix, wherein at least a portion of said delivery enhancing moieties are presented on the exterior surface of said self-sustaining body and/or on said emulsion droplets, said self-sustaining body being resistant to enteric degradation but capable of localization in the gastrointestinal tract of a subject without crossing the intestinal mucosa into the intestinal bloodstream.

26. (canceled)

27. The method of claim 25, wherein said emulsion is a solid-in-oil-in-water multiple emulsion, prepared by combining an oil, and a surfactant into a primary emulsion, wherein a solid form of the active agent is added to said primary emulsion under homogenization to yield said solid-in-oil-in-water multiple emulsion.

28. The method of claim 27, wherein said primary emulsion further comprises delivery enhancing moieties.

29. The method of claim 25, wherein said active agent emulsion comprises said active agent, an oil, a surfactant, and optional delivery enhancing moieties.

30. The method of claim 25, wherein said delivery enhancing moieties are provided in said polymer suspension and/or in said active agent emulsion.

31. (canceled)

32. The method of claim 30, wherein said delivery enhancing moieties in said polymer suspension are different from said delivery enhancing moieties in said active agent emulsion.

33. The method of 25, wherein said crosslinking comprises adding said mixture dropwise to a solution of crosslinking agent to yield said self-sustaining body.

34. The method of claim 33, wherein said adding comprises generating droplets of said mixture, rapidly freezing said droplets to yield frozen droplets, and dropping said frozen droplets into said solution of crosslinking agent.

35.-36. (canceled)

37. A microparticle for oral delivery of active agents to a subject, said microparticle comprising a self-sustaining body having an exterior surface, said self-sustaining body comprising an active agent encapsulated within emulsion droplets, wherein the emulsion droplets further comprise a plurality of delivery enhancing moieties, wherein at least a portion of said delivery enhancing moieties are presented on the exterior surfaces of said emulsion droplets.

38. (canceled)

39. The microparticle of claim 37, wherein said emulsion droplets comprise said active agent, an oil, a surfactant, and delivery enhancing moieties.

40.-43. (canceled)

44. A composition comprising a therapeutically-effective amount of a plurality of microparticles according to claim 37 dispersed in a pharmaceutically acceptable carrier.

45.-47. (canceled)
